(19) United States
(12) Patent Application Publication
(10) Pub. No.: US 2005/0171015 A1
(43) Pub. Date: Aug. 4, 2005

(54) Methods and agents for enhancing bone formation or preventing bone loss

(76) Inventors: Gerald R. Crabtree, Woodside, CA (US); Monte M. Winslow, Menlo Park, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/978,672

(22) Filed: Nov. 1, 2004

Related U.S. Application Data
(60) Provisional application No. 60/516,642, filed on Oct. 31, 2003.

(51) Int. Cl. A61K 38/18

(52) U.S. Cl. 514/12

(57) ABSTRACT

The present invention provides methods for (i) reducing loss of bone mass or bone density, (ii) increasing bone mass or bone density, (iii) maintaining bone mass or bone density, and/or (iv) reducing loss of calcium from bone, comprising: administering to a subject a therapeutically effective amount of an NFAT agonist. The method could be used for treating, preventing or delaying a bone condition. The invention further provides a method for promoting healing of bone fractures or bone defects comprising: administering to a subject a therapeutically effective amount of an NFAT agonist. Compositions comprising NFAT agonists can also be used for the in vitro or in vivo generation of bone tissue. The invention also provides screening methods for agents which promotes, maintains, or reduces the loss of, bone mass, and the use of such agents for therapeutic purposes.

### RELATED APPLICATIONS

This application claims priority to U.S. Provisional Application No. 60/516,642, filed Oct. 31, 2003, entitled “Methods and Compounds for Enhancing Bone Formation Or Preventing Bone Loss.” The entire teachings of the referenced application are incorporated by reference herein.

### STATEMENT REGARDING FEDERAL FUNDING

Certain work described herein was funded, in whole or in part, by Grant No. CA39612 from the National Institute of Health and by a grant from the Howard Hughes Medical Institute. The United States government has certain rights in this invention.

### BACKGROUND OF THE INVENTION

Bone is a living organ which undergoes remodeling throughout life. Remodeling results from the action of cells that form bone, osteoblasts, and cells that resorb bone, osteoclasts.

Bone Loss In Osteoporosis

Osteoporosis is a family of diseases characterized by the failure of the long bones to be remodeled. Osteoporosis affects an estimated 75 million people in Europe, United States, and Japan. Occurring predominantly in elderly women, osteoporosis is a major world health problem. Indeed most women over the age of 65 have clinically apparent osteoporosis. Osteoporosis has a great impact on the quality of life of many individuals. Osteoporosis leads to fractures, arthritis and significantly limits the lifestyle of many elderly people. Bone loss through osteoporosis leads to significant health care cost mostly by predisposing people to fractures and pain originating from defective bone remodeling in response to physical stress.

Bone is constantly being remodeled through bone resorption by osteoclasts and bone formation by osteoblasts. When these two processes are not balanced, and bone resorption is greater than bone formation, then osteoporosis results. After age 30 bone mass begins to decrease in both males and females. However after estrogen production decreases in menopausal woman the balance between bone formation and bone resorption is further skewed and bone loss becomes more rapid.

In addition to bone loss due to aging and changes in hormone balance during menopause, patients treated with steroids also develop osteoporosis. Studies on mice treated with steroids indicate that bone loss is rapid during the first year of steroid treatment and that this bone loss is due to decreased bone formation due to decreased numbers or activity of osteoblasts.

Bone loss and osteoporosis remain a serious medical and economic problem despite our current understanding of the fundamental cellular components of bone remodeling and the molecules that underlie bone resorption. Thus, there is a need for methods to treat bone loss in osteoporosis.

Bone Loss in Other Conditions

Bone loss is also important in other conditions, including but not limited to, acute and chronic renal failure, hyperparathyroidism, Paget's disease, periodontal disease and in healing of fractured bones.

Fractured bones heal readily in the young, but more slowly in the elderly. In all cases the time that is required for even common uncomplicated fractures to heal results in considerable morbidity, loss of time from work and inconvenience. Hence, a need exist for agents that might lead to more rapid healing of bone fractures at all ages.

Peridontal disease results in tooth loss and represents a significant problem in an aging population. Periodontal disease is another condition where bone loss is a major contributor and where significant health benefit could be achieved by agents that are able to prevent local bone loss.

Currently Available Treatments for Bone Loss

At this point there is no generally applicable treatment for bone loss. Treatment of menopausal woman with estrogens is well documented but is associated with many side effects and is not applicable to other situations of bone loss. Dietary calcium and weight bearing exercise when young are required to produce strong bones while insufficient dietary calcium and insufficient weigh bearing exercise when young are linked to osteoporosis latter in life.

NFAT Signaling in Mammalian Development

The NFATc (nuclear factor of activated T cells) family of transcription factors was first identified in T cells (Shaw et al., Science 241:202 (1988); Flanagan et al., Nature 352:803 (1991)). Subsequently this pathway has been shown to be import in cardiac development (Ranger et al., Nature 392:186 (1998); de la Pompa et al., Nature 392:182 (1998)), angiogenesis (Graef et al., Cell, 2001), neural development and function (Graef, Nature 105(7):863 (1999)), and osteoclast development (Ishida et al., J. Biol. Chem. 277(43):41147 (2002); Takayanagi et al., Dev. Cell 3(6):889 (2002)).

Before the present invention, it was found that NFATc1 was upregulated during osteoclast development and that NFATc1 was required for proper osteoclast development in vitro. (Ishida et al., J. Biol. Chem. 277(43):41147 (2002); Takayanagi et al., Dev. Cell 3(6):889 (2002))

NFAT is a transcription factor that remains cytosolic when phosphorylated. When cell stimulation results in an increase in intracellular calcium the heterodimeric serine/threonine phosphatase calcineurin is activated. Calcineurin dephosphorylates NFAT, which then translocates to the nucleus and binds to specific regions in the promoters of some gene. This nuclear import and activation of NFAT is opposed by rephosphorylation of NFAT by NFAT kinases and subsequent nuclear export.

Cyclosporin/FK506 Treatment and Osteoporosis

Patients treated with the immunosuppressive cyclosporin A (CsA) develop osteopenia. CsA binds to cyclophilin and the agent surface then binds to and inhibits calcineurin. CsA treated patients have an increased incidence of fractures and some experience calcineurin inhibitor induced pain syndrome (CIPS) (Grotz et al., Transpl. Int. 14(1):16 (2001)). The molecular mechanism underlying this bone loss has not been closely studied.

### SUMMARY OF THE INVENTION

In one embodiment, the invention relates to a method for (i) reducing loss of bone mass or bone density, (ii) increasing bone mass or bone density, (iii) maintaining bone mass or bone density and/or (iv) reducing loss of calcium from bone, comprising: administering to a subject a therapeutically effective amount of an NFAT agonist. As used in this patent specification, the term “bone mass” and “bone density” are used interchangeably.

In one embodiment, the invention relates to a method to regulate osteoblast activity or osteoclast activity comprising the use of an NFAT agonist. Osteoblast activity can be regulated by regulating the proliferation or function of osteoblasts. Osteoclast activity can be regulated by regulating the proliferation or function of osteoclasts. The function of osteoblasts and/or osteoclasts can be regulated directly or indirectly.

In one embodiment, the method is for the treatment of a bone condition or a bone defect.

In one embodiment, the bone condition being treated is osteoporosis. In one embodiment, the osteoporosis being treated is selected from the group consisting of: glucocorticoid-induced osteoporosis, hyperthyroidism-induced osteoporosis, immobilization-induced osteoporosis, heparin-induced osteoporosis and immunosuppressive-induced osteoporosis.

In another embodiment, the bone condition being treated is frailty, an osteoporotic fracture, a bone defect, childhood idiopathic bone loss, alveolar bone loss, mandibular bone loss, bone fracture, osteotomy, bone loss associated with periodontitis, or prosthetic ingrowth.

In yet another embodiment, the bone condition being treated is Paget's disease.

In another embodiment, the invention relates to method for promoting healing of bone fractures or bone defects comprising: administering to a subject a therapeutically effective amount of an NFAT agonist.

In another embodiment, the invention relates to method for bone tissue engineering comprising the use an NFAT agonist. In one embodiment the cells used for bone tissue engineering are treated with an NFAT agonist.

In one embodiment, the NFAT agonist is administered conjointly with an agent that increases bone mass or prevents the loss of bone mass. In one embodiment, the agent that increases bone mass is a growth factor, a mineral, a vitamin, a hormone, a prostaglandin, an inhibitor of 15-lipoxygenase, a bone morphogenic protein or another member of the TGF-beta superfamily which increases bone formation, an ACE inhibitor, a Hedghog protein, examethasone, calcitonin, or an active fragment thereof. In one embodiment, the agent that prevents the loss of bone mass is progestin, estrogen, an estrogen/progestin combinations, estrone, estriol, 17α- or 17β-ethynyl estradiol, SB242784, polyphosphonates, biphosphonates or an active fragment thereof.

In another embodiment, the invention relates to the use of an NFAT agonist as a medicament for (i) reducing loss of bone mass, (ii) increasing bone mass, (iii) maintaining bone mass and/or (iv) reducing loss of calcium from bone in a subject in need thereof. In another embodiment, the invention relates to the use of an NFAT agonist as a medicament for healing bone fractures or repairing bone defects in a mammal.

In one embodiment, the invention relates to a pharmaceutical composition comprising and NFAT agonist and a pharmaceutically acceptable carrier.

In another embodiment, the invention relates to a package pharmaceutical comprising the pharmaceutical composition described immediately above, in association with instructions for administering the composition to a subject for (i) reducing loss of bone density, (ii) increasing bone density, and/or (iii) reducing loss of calcium from bone. In yet another embodiment, the invention relates to a package pharmaceutical comprising the pharmaceutical composition described immediately above, in association with instructions for administering the composition to a subject for promoting healing of bone fractures.

In another embodiment, the invention relates to a method of determining whether an agent is an NFAT agonist which promotes, maintains, or reduces the loss of, bone mass or bone density comprising: (a) contacting the agent with a cell comprising NFAT and determining the location of NFAT within the cell in the presence and in the absence of the agent; wherein an increase of NFAT in the nucleus indicates that the agent is an NFAT agonist; and (b) further determining whether the agent promotes, maintains, or reduces the loss of, bone mass or bone density.

In another embodiment, the invention relates to a method of determining whether an agent is an NFAT agonist which promotes, maintains, or reduces the loss of, bone mass or bone density comprising: (a) contacting a cell expressing NFAT with an agent; and (b) determining the phosphorylation state of NFAT in the presence and absence of the agent; wherein a decrease in the phosphorylation of NFAT indicates that the agent is an NFAT agonist; and (b) further determining whether the agent promotes or maintains bone mass or bone density.

In another embodiment, the invention relates to a method of determining whether an agent is an NFAT agonist which promotes, maintains, or reduces the loss of, bone mass or bone density comprising: (a) contacting NFAT with a phosphatase under conditions that allow the dephosphorylation of NFAT in the presence and in the absence of an agent, and determining the phosphorylation state of NFAT, wherein an decrease in the phosphorylation indicates that the agent is an NFAT agonist. In one embodiment, the phosphatase is calcineurin; and (b) further determining whether the agent promotes, maintains, or reduces the loss of, bone mass or bone density.

In another embodiment, the invention relates to a method of determining whether an agent is an NFAT agonist which promotes, maintains, or reduces the loss of, bone mass or bone density comprising: (a) contacting NFAT with a kinase under conditions that allow the phosphorylation of NFAT in the presence and in the absence of an agent, and determining the phosphorylation state of NFAT, wherein an decrease in the phosphorylation indicates that the agent is an NFAT agonist; and (b) further determining whether the agent which promotes, maintains, or reduces the loss of, bone mass or bone density. In one embodiment, the kinase is GSK-3.

In another embodiment, the invention relates to a method of determining whether an agent is an NFAT agonist which promotes, maintains, or reduces the loss of, bone mass or bone density comprising: (a) transfecting a cell with an expression vector comprising a nucleic acid encoding a reporter gene operatively linked to an NFAT dependent transcriptional regulatory sequence; (b) incubating the cell in the presence and absence of an agent; (c) measuring the expression of the reporter gene; wherein an increase in the expression of the reporter gene indicates that the agent is an NFAT agonist; and (d) further determining whether the agent which promotes, maintains, or reduces the loss of, bone mass or bone density.

In another embodiment, the invention relates to a method of determining whether an agent is an NFAT agonist which increases osteoblast activity or decreases osteoclast activity comprising: (a) contacting the agent with a cell comprising NFAT and determining the location of NFAT within the cell in the presence and in the absence of the agent; wherein an increase of NFAT in the nucleus indicates that the agent is an NFAT agonist; and (b) further determining whether the agent increases osteoblast activity or decreases osteoclast activity.

In another embodiment, the invention relates to a method of determining whether an agent is an NFAT agonist which increases osteoblast activity or decreases osteoclast activity comprising: (a) contacting a cell expressing NFAT with an agent; and (b) determining the phosphorylation state of NFAT in the presence and absence of the agent; wherein a decrease in the phosphorylation of NFAT indicates that the agent is an NFAT agonist; and (b) further determining whether the agent increases osteoblast activity or decreases osteoclast activity.

In another embodiment, the invention relates to a method of determining whether an agent is an NFAT agonist which increases osteoblast activity or decreases osteoclast activity comprising: (a) contacting NFAT with a phosphatase under conditions that allow the dephosphorylation of NFAT in the presence and in the absence of an agent, and determining the phosphorylation state of NFAT, wherein an decrease in the phosphorylation indicates that the agent is an NFAT agonist; and (b) further determining whether the agent increases osteoblast activity or decreases osteoclast activity. In one embodiment, the phosphatase is calcineurin.

In another embodiment, the invention relates to a method of determining whether an agent is an NFAT agonist which increases osteoblast activity or decreases osteoclast activity comprising: (a) contacting NFAT with a kinase under conditions that allow the phosphorylation of NFAT in the presence and in the absence of an agent, and determining the phosphorylation state of NFAT, wherein an decrease in the phosphorylation indicates that the agent is an NFAT agonist; and (b) further determining whether the agent increases osteoblast activity or decreases osteoclast activity. In one embodiment, the kinase is GSK-3.

In another embodiment, the invention relates to a method of determining whether an agent is an NFAT agonist which increases osteoblast activity or decreases osteoclast activity comprising: (a) transfecting a cell with an expression vector comprising a nucleic acid encoding a reporter gene operatively linked to an NFAT dependent transcriptional regulatory sequence; (b) incubating the cell in the presence and absence of an agent; (c) measuring the expression of the reporter gene; wherein an increase in the expression of the reporter gene indicates that the agent is an NFAT agonist; and (d) further determining whether the agent increases osteoblast activity or decreases osteoclast activity.

In another embodiment, the invention relates to a method for identifying patients having, or at risk of having, a bone condition, which method comprises determining NFAT, or NFAT dependent, transcriptional activity levels in osteoblasts isolated from the patients, and identifying those patients having an abnormally low level of NFAT, or NFAT dependent, transcriptional activity as having or at risk of having a bone condition. In one embodiment, the NFAT is NFATc1.

In another embodiment, the invention comprises a method to screen for target genes that increase osteoblast activity or decrease osteoclast activity: (a) identifying a target gene whose expression is regulated by NFAT, and (b) determining whether the regulation of the expression of the target gene identified in step (a) increases osteoblast activity or decreases osteoclast activity. In one embodiment, the target gene whose expression is regulated by NFAT is identified by using an animal model expressing constitutively active NFAT or using an animal model where NFAT has been deactivated.

In another embodiment, the invention comprises a method to screen for target genes that reduce the lost of bone density or bone mass, and/or reduce loss of calcium from bone comprising: (a) identifying a target gene whose expression is regulated by NFAT, and (b) determining whether the regulation of the expression of the target gene identified in step (a) reduces loss of bone density, reduces the loss of bone mass, and/or reduces loss of calcium from bone in vivo.

In one embodiment the target gene whose expression is regulated by NFAT is identified by using an animal model expressing constitutively active NFAT or where the NFAT has been deactivated.

In another embodiment, the invention comprises a method to screen for agents that increase osteoblast activity or decrease osteoclast activity comprising: (a) identifying a target gene whose expression is regulated by NFAT, (b) determining whether the regulation of the expression of the target gene identified in step (a) increases osteoblast activity or decrease osteoclast activity, and (c) further identifying an agent that mimics or agonizes the activity of the target gene. In one embodiment, the agent is a small molecule. In one embodiment, the agent is a nucleic acid. In one embodiment, the agent is a polypeptide.

In another embodiment, the invention comprises a method to screen for agents that reduce the lost of bone mass or bone density, and/or reduce loss of calcium from bone comprising: (a) identifying a target gene whose expression is regulated by NFAT, (b) determining whether the regulation of the expression of the target gene identified in step (a) reduces loss of bone mass or bone density and/or reduces loss of calcium from bone in vivo, and (c) further identifying an agent that mimics or agonizes the activity of the target gene. In one embodiment, the agent is a small molecule. In one embodiment, the agent is a nucleic acid. In one embodiment, the agent is a polypeptide. In one embodiment the target gene whose expression is regulated by NFAT is identified by using an animal model expressing constitutively active NFAT. In another embodiment the target gene whose expression is regulated by NFAT is identified by using an animal model where NFAT has been deactivated.

The invention also comprises a method of using an agent identified in any of the screening methods described above to (i) reduce loss of bone mass or bone density, (ii) increase bone mass or bone density, (iii) maintain bone mass or bone density, and/or reduce loss of calcium from bone.

The invention also comprises a pharmaceutical composition comprising an agent identified in any of the screening methods described above and a pharmaceutically acceptable carrier.

In one embodiment, the invention comprises a method for (i) reducing loss of bone mass or bone density, (ii) increasing bone mass or bone density, (iii) maintaining bone mass or bone density and/or (iv) reducing loss of calcium from bone, comprising the use of an agent that regulates the activity of any one of the genes identified in FIGS. 9A, 9B or 10. In one embodiment, the method is to be carried out in a subject and comprises administering a therapeutically effective amount of the agent to the subject.

In another embodiment, the invention comprises a method for increasing osteoblast activity comprising the use of an agent that regulates the activity of any of the genes identified in FIGS. 9A, 9B or 10. In one embodiment, the method comprises the use of an agent that up-regulates the activity of at least one of the genes selected from the group consisting of: IGF binding protein 1, Wnt4, DSCR1, Egr2/Krox20, Tissue Plasminogen Activator (TPA), TGF-beta 1, BMP-1, PTH receptor, Frizzled 9, Stat3, cyclin F, nuclear protein 95, risc2/Cdt1, cdk4, cyclin D1 or cdc kinase subunit 1/Cks1. In one embodiment, the method comprises the use of an agent that down-regulates the activity of at least one of the genes selected from the group consisting of: Sfrp2, Pleitrophin/HB-GAM, Peirostin/facilin I-like, Asporin, Agiotensin Receptor type 2, COMP, Osteoglycin, and Dickkopf. In another embodiment, the method comprises the use of an agent that up-regulates the activity of at least one of the genes selected from the group consisting of: CCL8/MCP-2, CCL6/C10, CXCL 16, and CCL12/MCP-5. In one embodiment, the method is to be carried out in a subject and comprises administering a therapeutically effective amount of the agent to the subject.

In another embodiment, the invention comprises a method for decreasing osteoclast activity comprising the use of an agent that regulates the activity of any of the genes identified in FIG. 9B. In one embodiment, the method comprises the use of an agent that down-regulates the activity of at least one of the genes selected from the group consisting of: CCL8/MCP-2, CCL6/C10, CXCL 16, and CCL12/MCP-5. In one embodiment, the method is to be carried out in a subject and comprises administering a therapeutically effective amount of the agent to the subject.

In another embodiment, the invention comprises a method for increasing osteoblast activity comprising: (a) the use of an agent that up-regulates the activity of a gene that is regulated by NFAT and up-regulates osteoblast activity or proliferation and (b) the use of an agent that down-regulates the activity of a gene that is regulated by NFAT and up-regulates osteoclast activity.

In another embodiment, the invention comprises a method for increasing osteoblast activity comprising the use of: (a) an NFAT agonist, and (b) an agent that down-regulates the activity of a gene identified in FIG. 9B.

As used herein the term “agent” includes any compound or combinations of compounds, including, without limitation, small molecules, nucleic acid agents (including without limitation antisense nucleic acids, ribozymes and RNAi molecules) and polypeptides.

### BRIEF DESCRIPTION OF THE DRAWINGS

FIG. 1 illustrates that Eμ-tTA/c1nuc mice have a doxycycline suppressible increase in bone. (A) Hematoxylin Eosin stained femora of Eμ-tTA/NFATc1nuc and control mice with scale bars. (B) Radiographic analysis of femura and tibiae of Eμ-tTA/NFATc1nuc and control mice. (C) Treatment of Eμ-tTA/NFATc1nuc mice with doxycycline decreased bone density as shown in these radiographs of an Eμ-tTA/NFATc1nuc mice before doxycycline and then after 2, 4, and 8 weeks of doxycycline treatment.

FIG. 2 illustrates that doxycycline treatment prevents increase in bone in Eμ-tTA/NFATc1nuc mice. This along with the reversal of the phenotype in mice treated with DOX (FIG. 1) indicates that the phenotype is due to the expression of the transgene and not due to a non-specific integration effect.

FIG. 3 illustrates that adult mice have increased bone after transgene re-expression. Mice treated with doxycycline when young develop increased bone after doxycycline is stopped indicating that the NFAT pathway can increase bone formation in adult animals and has a role in both embryonic and adult bone formation. (Compare this X-ray to those in FIGS. 2 and 5.) FIG. 4 illustrates increased osteoblast activity in Eμ-tTA/NFATc1nuc mice. Eμ-tTA/NFATc1nuc mice have increased serum alkaline phosphatase (“ALP”) indicative of increased osteoblast activity. Serum ALP is a reliable marker for osteoblast function and bone formation.

FIG. 5 illustrates that mice expressing wild-type NFATc1 do not have increased bone indicating that a constitutively nuclear and constitutively active NFATc1 is required (and that increased wild type NFATc1 is not sufficient) to drive this high bone mass phenotype.

FIG. 6 illustrates that increased bone is driven by osteoblasts because transfer of Eμ-tTA/NFATc1nuc BM does not transfer phenotype. Wild-type mice (WT) were lethally irradiated and reconstituted with Eμ-tTA/NFATc1nuc bone marrow. The mice were either treated with doxycycline (+DOX) or left untreated. Osteoclasts are bone marrow derived and therefore the Eμ-tTA/NFATc1nuc mice→WT mice will have osteoclasts from the Eμ-tTA/NFATc1nuc bone marrow. The lack of bone phenotype in Eμ-tTA/NFATc1nuc mice (Tg ON) mice indicates that the phenotype is not osteoclast driven. These mice were analyzed three months after bone marrow transplantation.

FIG. 7 illustrates increased osteoblast numbers on Eμ-tTA/NFATc1nuc femura when compared to control mice. (Figure shows H&E stained femura from Eμ-tTA/NFATc1nuc and control mice.) FIG. 8 illustrates increased bone in two day old Eμ-tTA/NFATc1nuc mice. Alcian blue/alizarian red staining of two day old Eμ-tTA/NFATc1nuc mice and littermate control show increased rib width indicating increased bone formation during development. This indicates that the increased bone is a primary osteoblast driven phenotype. Images were taken at the same magnification.

FIG. 9A illustrates osteoblast functional genes and proliferations genes whose expression is misregulated in Eμ-tTA/NFATc1nuc mice. FIG. 9B illustrates monocyte chemoattractants and monocyte/osteoclast genes whose expression is misregulated in Eμ-tTA/NFATc1nuc mice. FIG. 9C illustrates that EμL-tTA/NFATc1nuc mice have increased TRAP staining. FIG. 9D illustrates that CCL8 is induced in a calcineurin-dependent manner after PMA/lonomycin stimulation. FIG. 10E illustrates that Wnt4 is induced in a calcineurin dependent manner after stimulation.

FIG. 10A-10H illustrates genes increased (FIG. 10A-10F) and decreased (FIG. 10G-10H) in P4 Eμ-tTA/NFATc1nuc mice compared to control.

FIG. 11 illustrates increased osteoblasts in Eμ-tTA/NFATc1nuc mice compared to control using Toluidine Blue staining.

FIG. 12 illustrates increases osteoblast proliferation in vivo in Eμ-tTA/NFATc1nuc mice using brdU labeling.

### DETAILED DESCRIPTION OF THE INVENTION

### Overview

The present invention is based on the observation that mice that express constitutively nuclear and active human NFATc1 have increased bone formation.

### Exemplary Embodiments

Exemplary Uses of NFAT Agonists

Compositions comprising NFAT agonists can be used to treat, prevent and alleviate bone conditions. The present invention provides a method for (i) reducing loss of bone mass, (ii) increasing bone mass, (iii) maintaining bone mass and/or (iv) reducing loss of calcium from bone, comprising: administering to a subject a therapeutically effective amount of an NFAT agonist. The method could be used for treating, preventing or delaying a bone condition. The invention further provides a method for promoting healing of bone fractures or bone defects comprising: administering to a subject a therapeutically effective amount of an NFAT agonist. Any of the above mentioned methods can involve the conjoint administration of an agent that increases bone mass or prevents the loss of bone mass.

The invention also provides for the use of an NFAT agonist as a medicament for treating, preventing or delaying a bone condition.

As used herein, the term “bone condition” includes any condition where it is desirable to increase bone mass or bone density and/or prevent the loss of bone mass or bone density. A bone condition includes any condition that increases osteoclast number, increases osteoclast activity, increases bone resorption, increases marrow fibrosis, or alters the calcium content of bone.

Non-limiting examples of bone conditions include metabolic bone conditions such as renal osteodystrophy, primary forms of osteoporosis (e.g., postmenopausal and senile osteoporosis), and secondary forms of osteoporosis that develop as a result of an underlying disease state. For example, osteoporosis can develop in patients that have endocrine disorders such as hyperparathyroidism, hypo- and hyperthyroidism, hypogonadism, hypercalcaemia due to malignancy, pituitary tumors, type I diabetes, or Addison's disease. Neoplasias such as multiple myeloma and carcinomatosis also can lead to development of osteoporosis. In addition, gastrointestinal problems such as malnutrition, malabsorption, hepatic insufficiency, and vitamin C or D deficiencies, and chronic administration of drugs such as anticoagulants, chemotherapeutics, corticosteroids, anticonvulsants, and alcohol can lead to development of osteoporosis.

Non-limiting examples of bone conditions also include osteonecrosis, osteoarthritis, rheumatoid arthritis, Paget's disease, osteogenesis imperfecta, chronic hyperparathyroidism, hyperthyroidism, Gorham-Stout disease, McCune-Albright syndrome, and alveolar ridge bone loss.

The term “bone condition” includes, without limitation, all conditions resulting in bone loss, including, cancers and tumors (such as osteosarcoma and multiple myeloma), renal disease (including acute renal failure, chronic renal failure, renal bone dystrophy and renal reperfusion injury), kidney disease, premature ovarian failure and other conditions.

Endocrine disorders, vitamin deficiencies and viral infections also can lead to development of bone conditions that can be treated with methods of the invention. An example of a bone condition caused by a nutritional disorder is osteomalacia, a nutritional disorder caused by a deficiency of vitamin D and calcium. It is referred to as “rickets” in children, and “osteomalacia” in adults. It is marked by a softening of the bones (due to impaired mineralization, with excess accumulation of osteoid), pain, tenderness, muscle wasting and weakness, anorexia, and overall weight loss. It can result from malnutrition, repeated pregnancies and lactation (exhausting or depleting vitamin D and calcium stores), and vitamin D resistance.

Bone conditions include conditions resulting from the treatment of a subject with drugs, for example the osteopenia resulting from the treatment with cyclosporin A or FK506.

Bone conditions also include bone fractures, bone trauma, conditions associated with post-traumatic bone surgery, post-prosthetic joint surgery, post-plastic bone surgery, post-dental surgery, bone chemotherapy, post-dental surgery and bone radiotherapy. Fractures include all types of microscopic and macroscopic fractures. Examples of fractures includes avulsion fracture, comminuted fracture, transverse fracture, oblique fracture, spiral fracture, segmental fracture, displaced fracture, impacted fracture, greenstick fracture, torus fracture, fatigue fracture, intraarticular fracture (epiphyseal fracture), closed fracture (simple fracture), open fracture (compound fracture) and occult fracture.

Other non-limiting examples of bone conditions include bone deformation, spinal deformation, prosthesis loosening, bone dysplasia, scoliosis, periodontal disease and defects, tooth repair, and fibrous osteitis.

The invention also provides a method for treating a subject with a therapeutically effective amount of an NFAT agonist wherein the subject is in need of bone repair following surgery, such as cranio-maxillofacial repair following tumor removal, surgical bone reconstruction following traumatic injury, repair of hereditary or other physical abnormalities, and promotion of bone healing in plastic surgery.

The invention also provides a method for treating a subject with a therapeutically effective amount of an NFAT agonist wherein the subject is in need of bone repair after receiving an implant (including joint replacements and dental implants), a prosthesis or a bone graft.

The invention also provides a method for treating a subject with a therapeutically effective amount of an NFAT agonist wherein the subject: a) is in need of increased bone density or bone healing; b) has undergone or is presently undergoing corticosteroid therapy, dialysis, chemotherapy for post menopausal bone loss, radiation therapy for cancer or hormone replacement therapy; c) is immobilized or subjected to extended bed rest due to bone injury; d) suffers from alcoholism, diabetes, hyperprolactinemia, anorexia nervosa, primary and secondary amenorrhea, or oophorectomy; e) suffers from renal failure; f) is 50 years or older; or g) is a female.

The invention also provides a method for treating a subject with a therapeutically effective amount of an NFAT agonist wherein the subject is affected by a disease selected from arterial calcification, ankylosing spondylitis, ossification of the posterior longitudinal ligament, myositis ossificans, diffuse idiopathic skeletal hyperostosis, calcific tendonitis, rotator cuff disease of the shoulders, bone spurs, cartilage or ligament degeneration due to hydroxyapatite crystal deposition, and chondrocalcinosis.

As used herein, “treating” means either slowing, stopping or reversing the progression of a condition.

As used herein, “subject” may include any animal, including mammals, capable of suffering from a bone condition. The subject can be a human, a dog, a cat, a primate, a porcine, a livestock animal, or any other mammal. In the preferred embodiment, the subject is a human being.

As used herein, “administering” may be effected or performed using any of the methods known to one skilled in the art. The agent may be administered by various routes including but not limited to aerosol, intravenous, oral or topical route. The administration may comprise intralesional, intraperitoneal, subcutaneous, intramuscular or intravenous injection; infusion; liposome-mediated delivery; topical, intrathecal, gingival pocket, per rectum, intrabronchial, nasal, transmucosal, inhalation, intestinal, oral, ocular or otic delivery. In a further embodiment, the administration includes intrabronchial administration, anal, intrathecal administration, transdermal delivery or liposome-mediated delivery. The NFAT agonists of the invention may be delivered locally via a capsule which allows sustained release of the agent or the peptide over a period of time.

The term administering also includes the delivery of an agent in an implantable matrix.

As used herein, “conjoint administration” means administration of two or more agents to a subject of interest as part of a single therapeutic regimen. The administration(s) can be either simultaneous or sequential, i.e., administering one agent followed by administering of a second (and/or a third one, etc.) at a later time, as long as the agents administered co-exist in the subject being treated, or at least one agent will have the opportunity to act upon the same target tissues of other agents while said target tissues are still under the influence of said other agents. In a preferred embodiment, agents to be administered can be included in a single pharmaceutical composition and administered together. In another preferred embodiment, the agents are administered simultaneously, including through separate routes. In yet another preferred embodiment, one or more agents are administered continuously, while other agents are administered only at predetermined intervals (such as a single large dosage, or twice a week at smaller dosages, etc.).

By the term “effective amount” or “therapeutically effective amount” of an NFAT agonist is meant an amount of an NFAT agonist sufficient to obtain the desired physiological effect, e.g., activation of osteoblasts, increase in osteoblast number, increase in bone formation, a decrease in osteoclasts number or the deactivation of osteoclasts. An effective amount of an NFAT agonist is determined by the care giver in each case on the basis of factors normally considered by one skilled in the art to determine appropriate dosages, including the age, sex, and weight of the subject to be treated, the condition being treated, and the severity of the medical condition being treated.

The effect of an NFAT agonist on a bone condition can be monitored. The monitoring step can include measuring calcium levels in a biological sample from the mammal, measuring levels of a marker of bone turnover in a biological sample from the mammal, or measuring bone mass and/or bone density in the mammal. The biological sample can be selected from the group consisting of blood, serum, plasma, bone, and urine. The marker of bone turnover can be selected from the group consisting of osteocalcin, bone specific alkaline phosphatase, type I C-terminal propeptide of type I collagen, deoxypyridinoline, and pyridinoline.

The bone mass or bone density in a subject can be monitored by means of bone density scan or other means of bone density evaluation. For example, bone mass and bone density can be monitored using dual-energy absorptiometry, quantitative computed tomography, quantitative ultrasound, radiography, or magnetic resonance imaging.

Compositions comprising NFAT agonists can be used in the in vitro or in vivo generation of bone tissue, such as from osteoblasts or any other type of cell which could give rise to bone tissue.

The invention also comprises a method for bone tissue regeneration comprising the use of an NFAT agonist. In one embodiment, the bone tissue regeneration comprises the use of cells transformed with an NFAT agonist or treated in vitro with an NFAT agonist. In another embodiment, the bone tissue regeneration further comprises the use of an absorbable biological material (such as an implantable matrix) as scaffolds for inducing the regeneration of bone tissue.

The invention comprises a method for treating bone conditions in a subject, comprising administering to the subject cells selected from the group consisting of embryonic stem cells, adult stem cells, osteoblastic cells, preosteoblastic cells, skeletal progenitor cells derived from bone, bone marrow or blood and mixtures thereof, wherein said cells have been treated in vitro with an effective amount of an NFAT agonist or have been transformed to express an NFAT agonist. In one embodiment, the treated cells are embedded within an implantable matrix comprising a prosthetic device or a surgical implant. The implantable matrix can be any matrix that allows the growth of bone tissue. The matrix can be made of synthetic polymers, ceramics (such as alumina and hydroxyapatite), native polymers (such as collagen or polysaccharide hyaluronic acid (HU)), or composites of ceramics and polymers. These matrices are well known in the art. See, e.g, Sharma and Elisseeff, “Engineering structurally organized cartilage and bone tissues,” Ann Biomed Eng. January 2004;32(1):148-59 and Ramoshebi et al., “Tissue engineering: TGF-beta superfamily members and delivery systems in bone regeneration,” Expert Rev Mol Med. Sep. 2, 2002;2002: 1-11, Shin et al., “Biomimetic materials for tissue engineering,” Biomaterials November 2003;24(24):4353-64, and Rose et al., “Delivery systems for bone growth factors—the new players in skeletal regeneration,” J. Pharm Pharmacol. April 2004;56(4):415-27, the contents of which are hereby incorporated by reference.

The invention also provides a method of producing bone at a bone defect site in vivo, the method comprising: implanting into the bone defect site a population of osteoblastic cells or osteoblast progenitor cells which have been cultured in vitro in the presence of an NFAT agonist. In one embodiment the cells are embryonic stem cells or adult stem cells.

The invention also provides a method for aiding the attachment of an implantable prosthesis to a bone site and for maintaining the long term stability of the prosthesis in a vertebrate, the method comprising coating selected regions of an implantable prosthesis with a NFAT agonist and implanting the coated prosthesis into the bone site, whereby such implantation promotes new bone formation.

The invention also provides a method for the ex vivo stimulation of bone mineralization, said method comprising culturing subject cells selected from the group consisting of embryonic stem cells, adult stem cells, osteoblastic cells, preosteoblastic cells and skeletal progenitor cells derived from bone, bone marrow, or blood, with an effective amount of an NFAT agonist; and incubating said cells for a time sufficient to allow for the promotion of nodule formation.

The invention also comprises a method for ex vivo skeletal tissue engineering, said method comprising culturing a population of cells in the presence of an NFAT agonist; and applying said cells to an implantable matrix and further incubating for a time sufficient for the cells to undergo osteogenesis; wherein the implantable matrix has bone tissue formation incorporating thereon and therein.

The invention also provides for a method for identifying patients having, or at risk of having, a bone condition, which method comprises determining NFAT, or NFAT dependent, transcriptional activity levels in osteoblasts isolated from the patients, and identifying those patients having an abnormally low level of NFAT, or NFAT dependent, transcriptional activity as having, or at risk of having, a bone condition. In one embodiment, the bone condition is osteoporosis. In one embodiment, the NFAT is NFATc1. Methods of determining transcriptional activity are well known in the art. For example, NFAT, or NFAT dependent, transcriptional activity can be determined by measuring the amount of NFAT that is localized in the nucleus of a cell, or by measuring the level of an intracellular second messenger responsive to activities dependent on an NFAT protein as described in more detail below.

Exemplary NFAT Agonists

The terms “NFAT,” “NF-AT,” “NFAT protein,” “NFATC,” and “NFATc” are used interchangeably herein. These terms refer to the family of nuclear factors of activated T cells. The GenBank Accession Numbers of exemplary human NFAT nucleic acids and polypeptides are provided in the following Table:

| NFAT | GenBank No. |
|---|---|
| NFATc1 | |
| NFATc | U08015 |
| NFATc.b | U59736 |
| NFATc2 | |
| NFAT1 | I38152 |
| NFATp1 | U43341 (isoform B); |
| | U43342 (isoform C) |
| NFATc3 | |
| NFAT4a | I38155 |
| NFAT4b | I38156 |
| NFAT4c | L41067 |
| NFATc4 | |
| NFAT3 | L41066, I38154 |
| NFATx | U14510 |
| NFATx2 | U85428 |
| NFATx3 | U85429 |
| NFATx4 | U85430 |

NFATc2 has also been referred to as NFIL2E, NFII-a, and NFATP.
NFATc1 has also been referred to as NFATC and NFAT2.
NFATc3 has also been referred to as NFAT4 and NFATX.
NFATc4 has also been referred to as NFAT3.

Other examples of NFAT genes and genes products can be found in GenBank, particularly accessions I80836, U36576, U36575, I60722, U02079, AF049606, AF087434, as well as PRF locus 2013343A, PIR locus S45262 and A48753. Exemplary NFAT polypeptides and nucleic acids are also disclosed in U.S. Pat. Nos. 6,388,052, 6,352,830, 6,312,899, 6,197,925, 6,171,781, 6,150,099, 6,096,515, and 5,837,840.

NFAT is a transcription factor that remains cytosolic when phosphorylated. When cell stimulation results in an increase in intracellular calcium the heterodimeric serine/threonine phosphatase calcineurin is activated. Calcineurin dephosphorylates NFAT, which then translocates to the nucleus and binds to specific regions in the promoters of some gene. This nuclear import and activation of NFAT is opposed by rephosphorylation of NFAT by NFAT kinases and subsequent nuclear export.

The term “NFAT agonist” as used herein refers to any molecule which activates or potentiates NFAT dependent gene transcription. Such agonists can accomplish this effect in various ways. For instance, NFAT agonists include molecules that can cause or promote a conformational change in an NFAT protein such that NFAT remains localized in the nucleus. For instance, one class of agonists will increase the amount of NFAT that is localized to the nucleus, such as by potentiating dephosphorylation of NFAT, or promoting conformational changes resulting from dephosphorylation of NFAT. Still another class of agonists can increase NFAT transcriptional activity by activating phosphatases that act on NFAT, such as calcineurin. Still other agonists inhibit phosphorylation of NFAT by inhibiting kinases that act on NFAT, such as GSK-3, PKA or DYRK1A. Constitutively active (e.g., constitutively nuclear) NFAT proteins or transcriptionally active fragments are also useful agonists. Other agonists are described herein and will be apparent to those skilled in the art.

NFAT agonists include, but are not limited to, molecules that: (1) interact directly in NFAT and modulate its nuclear translocation and activity; (2) interact directly with calcineurin and increase the dephosphorylation and/or activation of NFAT; (3) interact directly with calmodulin and increases the activity of calcineurin and the dephosphorylation and/or activation of NFAT; (4) stimulate an increase in intracellular calcium concentration which induces the activation of calcineurin and the dephosphorylation of NFAT; (5) bind to a cell surface receptor and induce an increase in intracellular calcium concentration which induces the activation of calcineurin and the dephosphorylation of NFAT; (6) interact with and inhibits GSK3 or other NFAT kinases which functions to increase the nuclear duration and activity of NFAT; (7) modify the DNA interaction of NFAT in order to increase NFAT dependent transcription; or (8) modify the interaction of NFAT with a nuclear partner that results in an increase in transcription. An NF-AT agonist may also be a molecule which increases or enhances the expression of NF-AT. An NF-AT agonist may also be a molecule which increases or enhances the expression of NF-AT.

In certain preferred embodiments, the NFAT agonists that are used in the subject methods are ones that promote nuclear localization of transcriptionally active NFAT proteins. In some embodiments, the method of the present invention utilizes molecules that change the allosteric conformation of NFAT, such that NFAT will be localized in the nucleus of a cell.

In certain embodiments, the methods of the present invention utilize NFAT agonists that enhance the dephosphorylation of NFAT. Such agonists include phosphatases such as calcineurin, and molecules that increase the activity or the expression of calcineurin.

In other embodiments, the methods of the present invention utilize NFAT agonists that inhibit the phosphorylation of NFAT. Such agonists include inhibitors of kinases such as GSK-3, PKA and DYRK1A (a priming kinase for nuclear GSK3).

In certain preferred embodiments, the NFAT agonist activates NFATc1-dependent gene transcription.

The NFAT agonists that are used in the subject methods may be small organic molecules or other biological molecules such as nucleic acids and proteins. The NFAT agonists that are used in the subject methods may be applied to the target cells, e.g., formulated to be taken up by the target osteoblasts, or introduced into the target cells by techniques known in the art. Such techniques for targeting the NFAT agonist to the target cells include, without limitation, the use of fusion or chimeric proteins including peptides such as the N-terminal sequence of HIV, a fragment of antennapedia, and a fragment C of tetanus toxin (Francis et al., Brain Res. 995(1):84-96 (2004). Other delivery vehicles are described in the art. See, e.g., Young et al, “Muscle-based gene therapy and tissue engineering to improve bone healing,” Clin Orthop. October 2002 (403 Suppl):S243-51, Kirker-Head CA, “Potential applications and delivery strategies for bone morphogenetic proteins,” Adv Drug Deliv Rev. Sep. 15, 2000;43(1):65-92, and Rose et al., “Delivery systems for bone growth factors—the new players in skeletal regeneration,” J. Pharm Pharmacol. April 2004;56(4):415-27the contents of which are hereby incorporated by reference.

In one embodiment, the NFAT agonist is a constitutively active NFAT protein. A constitutively active NFAT protein may be a naturally occurring protein or a mutant. Constitutive active NFAT proteins are known in the art. See, e.g., Neal and Clipstone, J. Biol. Chem. 278(19):17246-54 (2003); Porter and Clipstone, J. Immunol. 168(10):4936-45 (2002); Monticelli and Rao, Eur. J. Immunol. 32(10):2971-78 (2002); Plyte et al., J. Biol. Chem. 276(17):14350-58 (2001). Nucleic acids encoding constitutively active forms of NFAT can be introduced into the target cell by techniques known in the art, such as gene therapy.

Further, constitutively active forms of NFAT can be applied to target cells using delivery techniques such as liposomes, or by forming fusion or chimeric proteins of a constitutively active NFAT protein that includes a fusogenic peptide such as the N-terminal sequence of HIV-TAT protein, a fragment of the antennapedia III protein or fragment C of tetanus toxin (Francis et al., Brain Res. 995(1):84-96 (2004). Other delivery vehicles are described in the art.

In certain embodiments, the methods of the present invention utilize NFAT agonists that enhance the activity of calcineurin. For instance, the activity of calcineurin can be enhanced or increased through introduction of a gene that expresses calcineurin or a protein that upregulates the expression of calcineurin or a protein that prevents the down-regulation of calcineurin (such as MCIPs). The introduction of a gene (an endogenous gene that has been altered, or a gene originally isolated from a different organism, for example) into cells, either in vitro or in a patient, can be accomplished by any of several known techniques, for example, by vector mediated gene transfer, as by amphotropic retroviruses, calcium phosphate, or liposome fusion, for example.

A gene intended to have an effect on osteoblasts in a host mammal can be delivered to isolated osteoblast cells by the use of viral vectors comprising one or more nucleic acid sequences encoding the gene of interest. Generally, the nucleic acid sequence has been incorporated into the genome of the viral vector. In vitro, the viral vector containing the nucleic acid sequences encoding the gene can be contacted with a cell and infection can occur. The cell can then be used experimentally to study, for example, the effect of the gene on growth of osteoblasts cells in vitro or the cells can be implanted into a patient for therapeutic use. The cells to be altered by introduction or substitution of a gene can be present in a biological sample obtained from the patient and used in the treatment of disease, or can be obtained from cell culture and used to dissect developmental pathways of arteries and veins in in vivo and in vitro systems.

After contact with the viral vector comprising a nucleic acid sequence encoding the gene of interest, the treated osteoblasts can be returned or re-administered to a patient according to methods known to those practiced in the art. Such a treatment procedure is sometimes referred to as ex vivo treatment. Ex vivo gene therapy has been described, for example, in Kasid et al., Proc. Natl. Acad. Sci. USA 87:473 (1990); Rosenberg et al., New Engl. J. Med. 323:570 (1990); Williams et al., Nature 310:476 (1984); Dick et al., Cell 42:71 (1985); Keller, et al., Nature 318:149 (1985); and Anderson et al., U.S. Pat. No. 5,399,346 (1994).

Generally, viral vectors which can be used therapeutically and experimentally are known in the art. Examples include the vectors described by Srivastava A., U.S. Pat. No. 5,252,479 (1993); Anderson et al., U.S. Pat. No. 5,399,346 (1994); Ausubel et al., “Current Protocols in Molecular Biology”, John Wiley & Sons, Inc. (1998). Suitable viral vectors for the delivery of nucleic acids to cells include, for example, replication defective retrovirus, adenovirus, parvovirus (e.g., adeno-associated viruses), and coronavirus. Examples of retroviruses include avian leukosis-sarcoma, mammalian C-type, B-type viruses, lentiviruses (Coffin, J. M., “Retroviridae: The Viruses and Their Replication”, In: Fundamental Virology, Third Edition, B. N. Fields, et al., eds., Lippincott-Raven Publishers, Philadelphia, Pa., (1996)). The mechanism of infectivity depends upon the viral vector and target cell. For example, adenoviral infectivity of HeLa cells occurs by binding to a viral surface receptor, followed by receptor-mediated endocytosis and extrachromasomal replication (Horwitz M. S., “Adenoviruses” In: Fundamental Virology, Third Edition, B. N. Fields et al., eds., Lippincott-Raven Publishers, Philadelphia, Pa., (1996)).

Instead of gene therapy, a calcineurin protein or a molecules that activates a calcineurin protein can be applied to the target cells, e.g., formulated to be taken up by osteoblasts.

In one embodiment, the methods of the present invention utilize NF-AT agonists that inhibit a modulatory calcineurin-interacting protein (MCIP). In one embodiment, the NF-AT agonist to be used in the claimed methods is the pyridine activator of myocite hypertrophy (“PAMH”) disclosed in Bush et al., PNAS, 101(9):2870-2875 (2004).

Another embodiment of the invention relates to methods for decreasing the level of NFAT phosphorylation in a mammal by administering an agent which: down-regulates gene expression of GSK3 or other kinases that phosphorylate NFAT proteins (such as PKA or DYRK1A); inhibit the expression of GSK3 (such as antisense RNAi constructs); acts upstream of GSK3 and down-regulate its expression, stability or activation as a kinase; as well as pharmacological inhibitors of GSK3 (such as small organic molecules that bind to an inhibit the kinase activity of GSK3). A preferred agent is a nucleic acid, such as an antisense nucleic acid or an RNA interference (RNAi) construct. Optionally, the agent is a small molecular compound. In certain cases, bone formation may be promoted when the agent reduces gene expression or function of GSK3. International Patent Applications Publication Numbers WO 02/062387, WO 00/21927, WO 00/386755 WO 01/09106 and WO 01/74771 (SmithKline Beecham PLC), W098/16528 and U.S. application Nos. 2004/0024040 and 2004/0019052 disclose certain agents useful as GSK-3 inhibitors. The teachings of those publications are incorporated by reference herein

For example, the invention contemplates the use of antisense nucleic acid corresponding to a portion of a gene encoding a GSK3 polypeptide, which antisense decreases the level of expression of the GSK3 protein. Such an antisense nucleic acid can be delivered, for example, as an expression plasmid which, when transcribed in the cell, produces RNA which is complementary to at least a unique portion of the cellular mRNA which encodes GSK3. Alternatively, the construct is an oligonucleotide which is generated ex vivo and which, when introduced into the cell causes inhibition of expression by hybridizing with the mRNA and/or genomic sequences encoding GSK3. Such oligonucleotide probes are optionally modified oligonucleotides which are resistant to endogenous nucleases, e.g., exonucleases and/or endonucleases, and is therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., Biotechniques 6:958-976 (1988); and Stein et al., Cancer Res. 48:2659-2668 (1988).

In certain aspects, the invention relates to the use of RNA interference (RNAi) to effect knockdown of GSK3 or other kinases which phosphorylate NFAT. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. RNAi provides a useful method of inhibiting gene expression in vitro or in vivo. RNAi constructs can comprise either long stretches of dsRNA identical or substantially identical to the target nucleic acid sequence or short stretches of dsRNA identical to substantially identical to only a region of the target nucleic acid sequence.

Optionally, the RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the method has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The subject RNAi constructs can be “small interfering RNAs” or “siRNAs.” These nucleic acids are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, e.g., Heidenreich et al., Nucleic Acids Res., 25:776-780 (1997); Wilson et al., J. Mol. Recog. 7:89-98 (1994); Chen et al., Nucleic Acids Res. 23:2661-2668 (1995); Hirschbein et al., Antisense Nucleic Acid Drug Dev. 7:55-61 (1997)). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

In some cases, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

The RNAi construct can also be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response.

Alternatively, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev. 16:948-58 (2002); McCaffrey et al., Nature 418:38-9 (2002); McManus et al., RNA 8:842-50 (2002); Yu et al., Proc. Natl. Acad. Sci. USA 99:6047-52 (2002)). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In other aspects, the method relates to the use of ribozyme molecules designed to catalytically cleave an mRNA transcripts to prevent translation of mRNA, such as GSK3 mRNA (see, e.g., PCT International Publication W090/11364, published October 4, 1990; Sarver et al., Science 247:1222-1225 (1990); and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach 1988, Nature, 334:585-591. The ribozymes of the present invention also include RNA endoribonucleases (hereinafter “Cech-type ribozymes”) such as the one which occurs naturally in Tetrahymena thermophila (known as the IVS or L-19 IVS RNA) and which has been extensively described (see, e.g., Zaug et al., Science, 224:574-578 (1984); Zaug and Cech, Science, 231:470-475 (1986); Zaug et al., Nature, 324:429-433 (1996); published International patent application No. WO88/04300 by University Patents Inc.; Been and Cech, Cell, 47:207-216 (1986)).

In a further aspect, the invention relates to the use of DNA enzymes to inhibit expression of GSK3. DNA enzymes incorporate some of the mechanistic features of both antisense and ribozyme technologies. DNA enzymes are designed so that they recognize a particular target nucleic acid sequence, much like an antisense oligonucleotide; however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

In addition to affecting the levels or activity of calcineurin and GSK3 using biological macromolecules, small organic molecules can also be used to increase the activity of calcineurin or decrease the activity of GSK3 in a patient, either in the affected tissues specifically or throughout a patient's tissues.

Agents that activate, agonize, or mimic the activity of calcineurin are NFAT agonists. These agents include, but are not limited to, calcium ionophores, such as A23187 and ionomycin, angiotensin II, phenylephrine, 1% fetal bovine serum, carbachol, cholecystokinin (including the 26-33 fragment), and cholinergic agonists such as carbamylcholine.

Agents that antagonize, inhibit, or suppress the activity of GSK3 are also NFAT agonists. These agents include, but are not limited to, insulin, wnt proteins, MAPKAP-K1 (RSK), protein kinase B (Akt), paullones such as alsterpaullone (Leost et al., Eur. J. Biochem. 267:5983-94 (2000)), growth factor (GF), epidermal growth factor (EGF), lithium chloride, maleimides such as Ro 31-8220, SB 216763, and SB 415286, aloisines such as aloisines A and B, p70 ribosomal S6 kinase 1 (S6K1), cyclic AMP analogs and agonists, hymenialdisines such as dibromohymenialdisine, indirubins such as 5,5′dibromo-indirubin, muscarinic antagonists such as AF 150 and AF102B, and Frequently rearranged in advanced T-cell lymphomas 1 (FRAT1) (including the 188-226 fragment). GSK3 has recently been reviewed, S. Frame and P. Cohen, Biochem. J. 359:1-16 (2001), and additional information about GSK3 has been surveyed, B. W. Doble and J. R. Woodget, J. Cell Sci. 116:1175-1186 (2003).

In one embodiment, the NFAT agonists are modified to enhance their potency. In one embodiment, the NFAT agonists are modified in a way that results in the agent been sequestered in a bone. In one embodiment, the NFAT agonist is modified by the covalent attachment of moieties related to tetracycline or other molecules that are concentrated in the bone and by virtue of their concentration lead to enhanced effects in bone. Biphosphonates and antibodies or other binding proteins which interact specifically with surface molecules on bone tissue cells may also be used to target the NFAT agonists of the invention to bone tissue.

In one embodiment the claimed methods use an NFAT agonist that is an agonist of peroxisome proliferator-activated receptor-gamma (“PPARgamma”). PPAR gamma agonists are well known to those of skill in the art and include, for example, thiozolidinediones (TZD). Particularly preferred PPARgamma agonists include, but are not limited to rosiglitazone, troglitazone (Resulin), farglitazar, phenylacetic acid, GW590735, GW677954, Avandia, Avandamet (avandia+metformin), ciglitazone, 15 deoxy prostaglandin J2 (15PGJ2), 15-deoxy-delta12,14 PGJ2, GW-9662, MCC-555 (disclosed in U.S. Pat. No. 5,594,016), analogues thereof and the like. PPAR gamma agonists include thiazolidinedione derivatives such as pioglitazone [(±)[[4-[2-(5-ethyl pyridinyl)ethoxy]phenyl]methyl]-2,4thiazolidinedione], troglitazone [(±)[[4-[(3,4-dihydro hydroxy-2,5,7,8-tetramethyl-2H benzopyran yl)methoxy]phenyl]methyl]-2,4-thiazolidinedione], ciglitazone [5-[[4-[(lmethylcyclohexyl)methoxy]phenyl]methyl]-2,4-thiazolidinedione, rosiglitazone [(±)[4-[2-[N-methyl-N-(2-pyridyl)amino]ethoxy]benzyl]-2,4-thiazolidinedione] and other 2,4thiazolidinedione derivatives as well as pharmaceutically suitable acid addition salts thereof. Other PPARgamma agonist include: S)-2-ethoxy-3-[4-(2-{4-methanesulphonyloxyphenyl}ethoxy-)phenyl]propanoic acid, WY-14643, clofibrate, fenofibrate, bezafibrate, GW 9578, englitazone (CP-68722, Pfizer), proglitazone, BRL-49634, KRP-297, JTT-501, SB 213068, GW 1929, GW 7845, GW 0207, L-796449, L-165041, GW 2433, GL-262570 (Glaxo Welcomes), darglitazone (CP-86325, Pfizer, isaglitazone (MIT/J&J), JTT-501 (JPNT/P&U), L-895645 (Merck), R-119702 (Sankyo/WL), NN-2344 or balaglitazone (Dr. Reddy/NN), or YM-440 (Yamanouchi). Other PPARgamma agonists include AZ-242/tesaglitazar (Astra/Zeneca; as described: in B. Ljung et. al., J. Lipid Res., 2002, 43, 1855-1863), GW-409544 (Glaxo-Wellcome), KRP-297/MK-767 (Kyorin/Merck; as described in: K. Yajima et. al., Am. J. Physiol. Endocrinol. Metab., 2003, 284: E966-E971) as well as those disclosed by Murakami et al, “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998) or the agents (from Bristol-Myers Squibb) described in U.S. Pat. No. 6,414,002. Other PPARgamma agonist include GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchrom-ane-2-carboxylic acid (disclosed in U.S. Ser. Nos. 60/235,708 and 60/244,697).

Additional NFAT agonists can be identified using the screening assays described immediately below.

Exemplary Screening Assays

The invention provides for screening assays to identify NFAT agonists. More particularly, the invention provides for screening assays to identify NFAT agonists which augment or maintain bone mass or bone density, reduce the loss of bone mass or bone density, increase bone density, reduce loss of calcium from bone, regulate osteoclast activity, regulate osteoblast activity, promote bone regeneration, or aid in bone tissue engineering. The screening assays of the invention permit the ready screening of large numbers of small synthetic molecules, natural products, peptides and proteins. The molecules to be screened are held in collections by numerous pharmaceutical companies, are often created as a result of combinatorial chemistry or are modifications of existing drugs, peptides and proteins. The screening assays can be in vivo or in vitro and can be cell based or based on a cell free format.

The invention provides screening assays for identifying agents which inhibit phosphorylation of a NFAT protein or increase depohosphorylation of an NFAT protein. In this regard, an NFAT agonist is an agent which either inhibits phosphorylation of an NFAT protein, or potentiates dephosphorylation of an NF-AT protein. In certain embodiments of the assay, it may be desirable to directly detect changes in phosphorylation of an NFAT protein.

In one embodiment, the assay is an in vitro assay. In one embodiment, the assay comprises contacting a non-phosphorylated, or partially phosphorylated NFAT protein with a cell extract, or with one or more purified kinases, such as GSK-3, PKA or DYRKIA, and other necessary components of an in vitro kinase assay, including a source of phosphate and with or without a test agent and under conditions under which phosphorylation of NFAT occurs. The comparison of the state of phosphorylation of NFAT in the presence and in the absence of a test agent will indicate whether the test agent decreases or inhibits the phosphorylation of NFAT.

In another embodiment, the kinase assay is an in vivo kinase assay. The assay can comprise incubating a cell expressing non-phophorylated or partially phosphorylated NFAT, e.g., an activated T cell, with a test agent and comparing the state of phosphorylation of NFAT in the presence and in the absence of the test agent. A variation in the state of phosphorylation will indicate that the test agent is capable of modulation phosphorylation of NFAT. The state of phosphorylation of NFAT can be determined by, e.g., by performing the incubation of the cells in the presence of labeled, e.g., radioactive, phosphate (e.g., ATP), and determining the amount of label present in an immunoprecipitate with an NFAT specific antibody. Alternatively, the state of phosphorylation can be performed by Western blot analysis, optionally coupled with immunoprecipitations.

In another embodiment; the invention provides screening assays for identifying agents which increase dephosphorylation of NFAT, such as activators of calcineurin-mediated dephosphorylation of an NFAT protein. In one embodiment, the assay comprises incubating a phosphorylated NFAT protein with a cell extract or with one or more phosphatases, e.g., calcineurin, in conditions under which the NFAT polypeptide can be dephosphorylated, and a test agent. The NFAT protein can be phosphorylated in vitro with PKA and optionally GSK-3, or it can be phosphorylated with a cell extract. NFAT can also be isolated from or present in a cell extract. The comparison of the state of phosphorylation of NFAT after a phosphatase reaction in the presence and in the absence of a test agent will indicate whether the test agent is capable of increasing dephosphorylation of NFAT, and therefore be a candidate NFAT agonist. The state of phosphorylation of NFAT can be determined as described above.

In yet another embodiment, the drug screening assay is derived to include a whole cell expressing an NFAT protein. For instance, the level of an intracellular second messenger responsive to activities dependent on an NFAT protein can be detected. For example, in various embodiments the assay may assess the ability of test agent to cause changes in or expression of genes whose transcription is dependent on an NFAT protein. By detecting changes in intracellular signals, such as alterations in second messengers or gene expression, candidate agonists of NFAT dependent signaling can be identified.

By selecting transcriptional regulatory sequences from target genes, e.g., NFAT dependent transcriptional control elements, and operatively linking such promoters to a reporter gene, the present invention provides a transcription based assay which is sensitive to the ability of a specific test agent to influence signaling pathways dependent on an NFAT protein.

In an exemplary embodiment, the subject assay comprises detecting, in a cell-based assay, change(s) in the level of expression of a reporter gene controlled by a transcriptional regulatory sequence responsive to signaling by an NFAT protein. Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on signaling by the NFAT protein. Expression of the reporter gene, thus, provides a valuable screening tool for the development of agents or compounds that act as agonists or antagonists of NFAT protein-dependent signaling. The reporter gene may be a luciferase or lacZ gene. The use of transcription based assays is well known in the art.

The invention further provides screening assays for identifying agents which increase nuclear localization of an NFAT protein. In one embodiment, the screening assay measures the movement of NFAT from the cytoplasm to the nucleus of a cell, such as an osteoblast. This screening assay allows the identification of NFAT agonists that prevent nuclear exit of NFATc proteins or enhance nuclear import. In one embodiment the NFAT protein is labeled with a molecule that allows the NFAT to be readily visualized. In one embodiment, the NFAT is labeled with GFP (green fluorescent protein).

In one embodiment, the screening assay measures the movement of NFAT from the cytoplasm to the nucleus of a cell by detecting an allosteric change in NFAT detectable by FRET (Fluorescent Resonance Energy Transfer).

The invention further provides screening assays for identifying agents which increase nuclear localization of an NF-AT protein. The screening assays can be in vivo or in vitro and can be cell based or based on a cell free format. In a preferred embodiment, the assays allow the identification of agents which increase NF-AT translocation across the nuclear membrane. In certain embodiments, the translocation of NF-AT across the nuclear membrane is detected using immunofluorescence.

After an NFAT agonist has been identified using any of the methods described above, the agent can be further tested for determining whether it augments or maintain bone mass or bone density, reduces the loss of bone mass or bone density, increases bone mass or bone density, reduces loss of calcium from bone, regulates osteoclast activity, regulates osteoblast activity, promotes bone regeneration, or aids in bone tissue engineering.

Pharmaceutical Compositions Comprising NFAT Agonists

The invention also comprises a pharmaceutical composition comprising a therapeutically effective amount of an NFAT agonist and a pharmaceutically acceptable carrier.

The invention also comprises a package pharmaceutical comprising a pharmaceutical composition comprising a therapeutically effective amount of an NFAT agonist and a pharmaceutically acceptable carrier, in association with instructions for administering the composition to a subject.

In one embodiment, the NFAT agonist is calcineurin or an activator of calcineurin. In another embodiment, the NFAT agonist is an inhibitor of GSK3, PKA or DYRK1A. In another embodiment, the NFAT agonist is a constitutively active NFAT protein.

In certain embodiments, the pharmaceutical composition comprises an NFAT agonist and other components. The additional component may be another NFAT agonist, an osteoclast inhibitor, an osteoblast activator, or another agent.

In one embodiment, the pharmaceutical composition of the invention comprises an NFAT agonist and another agent that increases bone mass or bone density, or prevents the loss of bone mass or bone density. Agents that increase bone mass or bone density include, but are not limited to, growth factors (such as IGF-1, IGF-2, macrophage growth factor, platelet derived growth factor, fibroblast growth factor, epidermal growth factor, transforming growth factor and connective tissue growth factor), minerals (such as calcium, aluminum strontium and fluoride), vitamins (such as Vitamin D3), natural and synthetic hormones (such as parathyroid hormone (PTH), parathyroid hormone related protein (PTHrP)), prostaglandins (such as PGD1, PGD2, PGE2, PGE1 and PGF2), inhibitors of 15-lipoxygenase, dexamethasone and bone morphogenic proteins (such as BMP-2, BMP-4 and BMP-7), ACE inhibitors, Hedgehog proteins (such as Sonic Hedgehog and Indian Hedgehog), calcitonin, and active fragments of any of the above mentioned proteins. Agents that prevent bone loss include, but are not limited to, progestins, estrogen, estrogen/progestin combinations, estrone, estriol, 17α- or 17β-ethynyl estradiol, SB242784, polyphosphonates, biphosphonates and active fragments of any of the above mentioned proteins. Commercially available bisphosphonates include: etidronate, clodronate, tiludronate, alendronate, pamidronate and ibandronate. Agents that increase bone mass or prevent loss of bone mass are well known in the art.

In one embodiment, the pharmaceutical composition of the invention comprises an NFAT agonist and another agent that targets the NFAT agonist to the bone. For example, tetracycline and diphosphonates are known to bind bone mineral, particularly at zones of bone remodeling, when they are provided systemically in a mammal. Alternatively, an antibody or other biding protein that interacts specifically with a surface molecule on bone tissue cells also may be used.

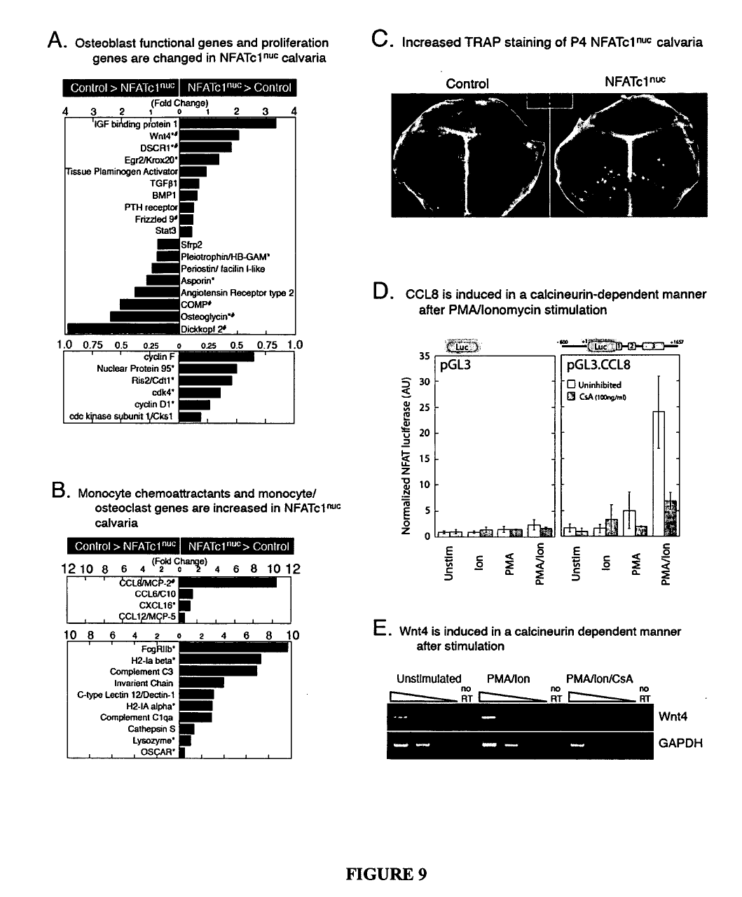

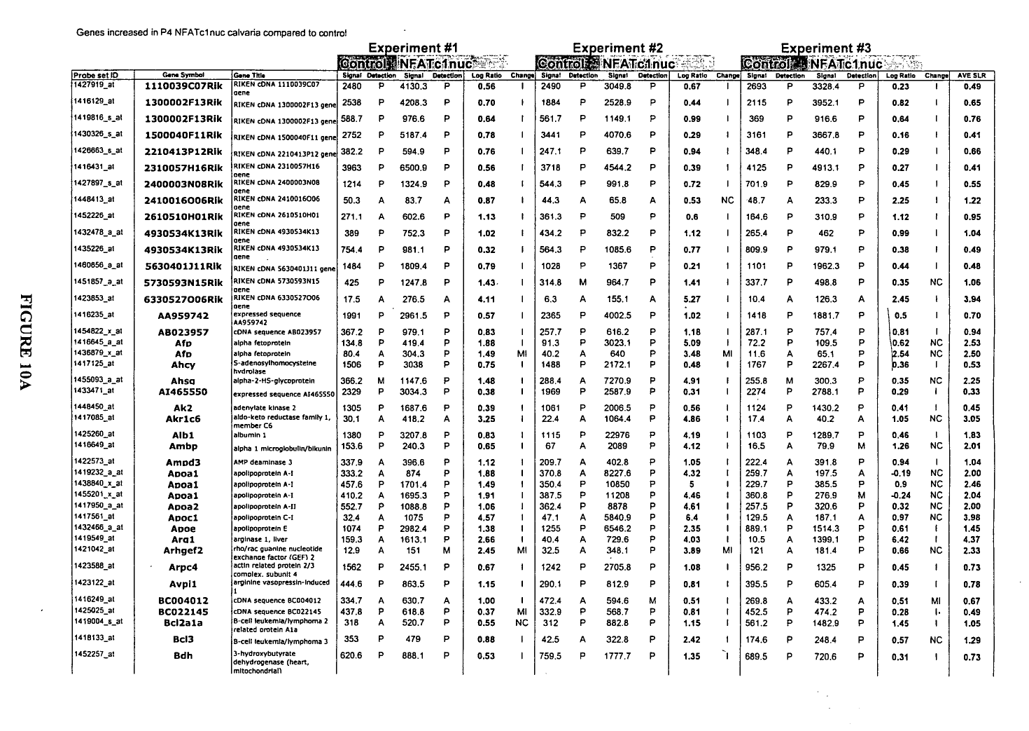

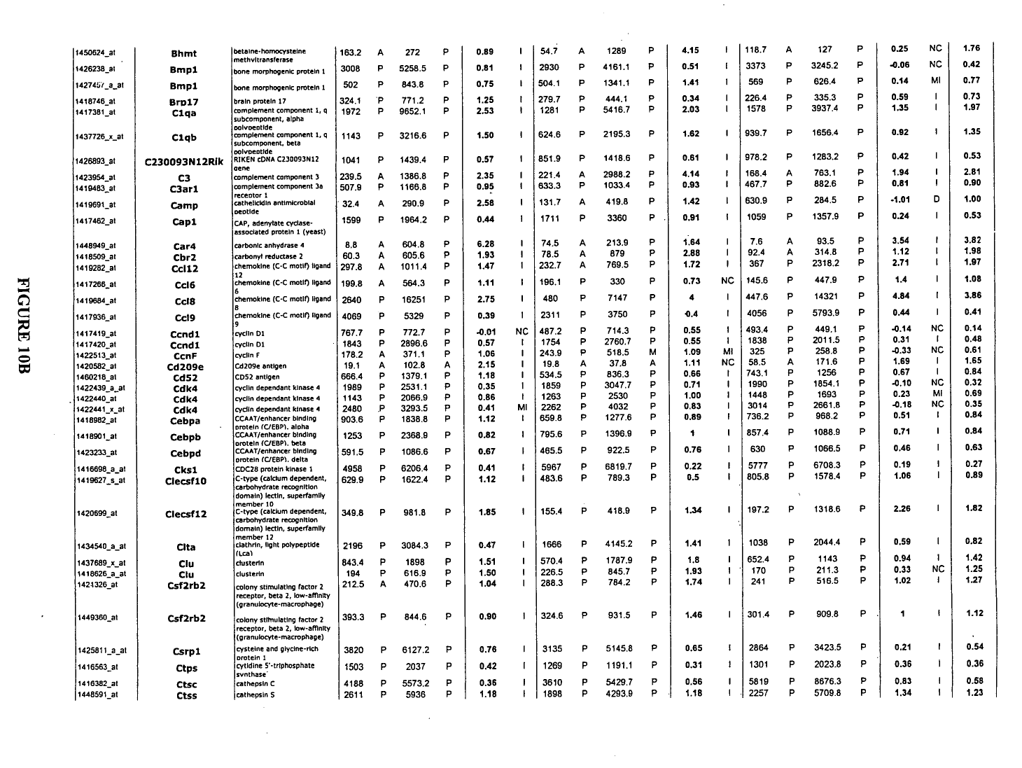

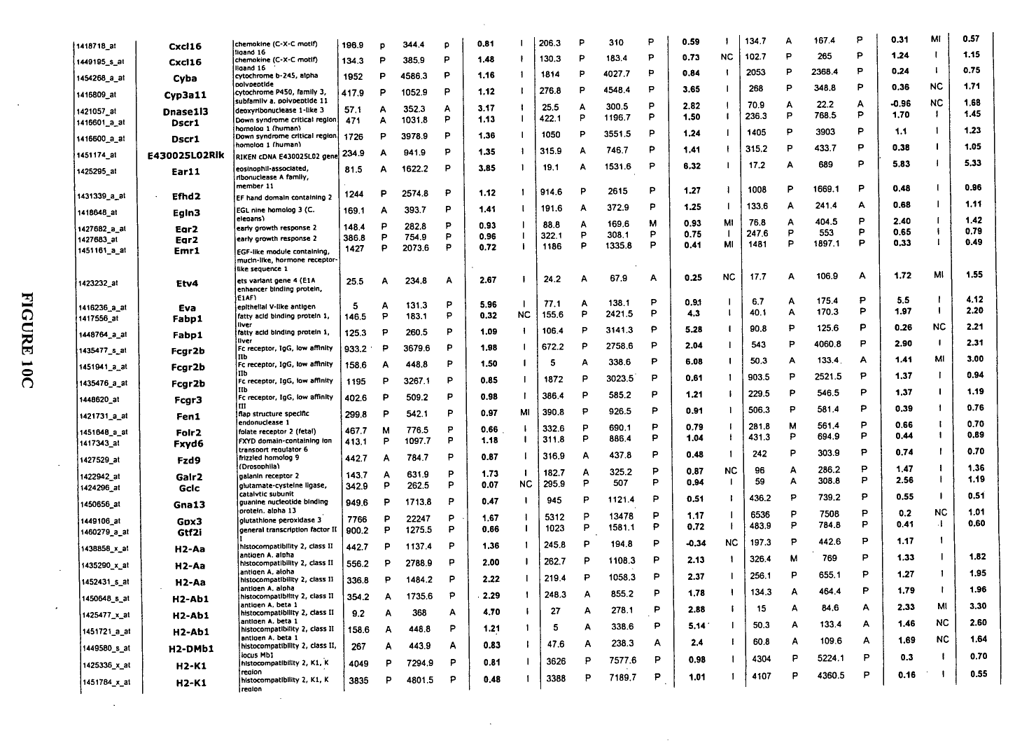

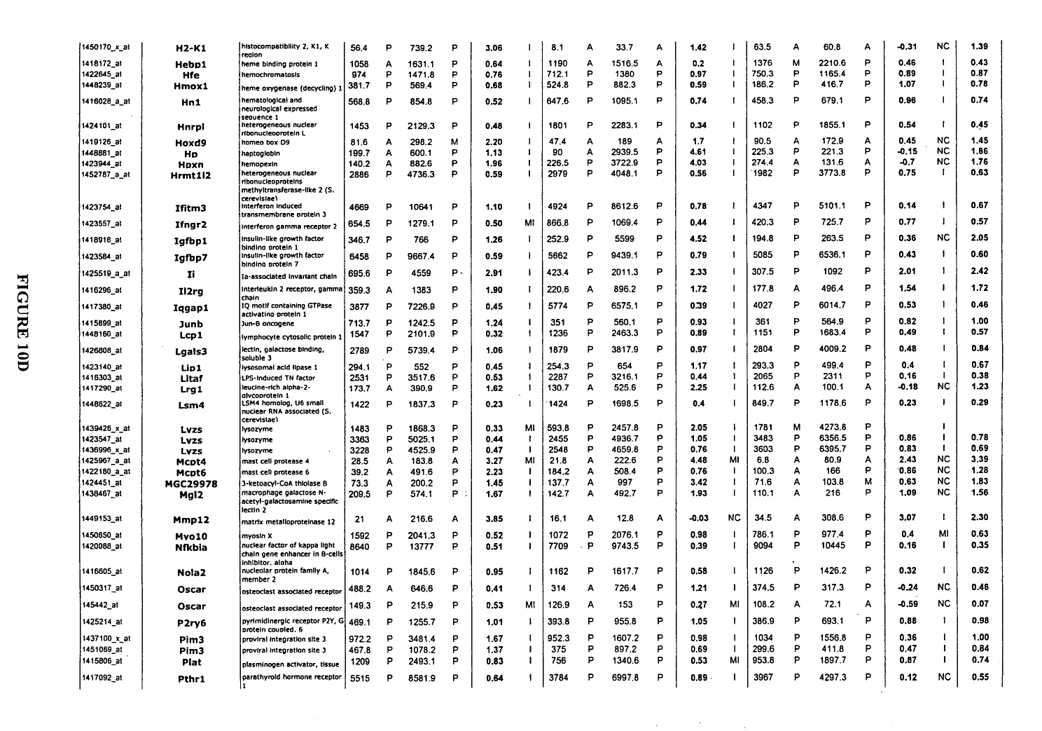

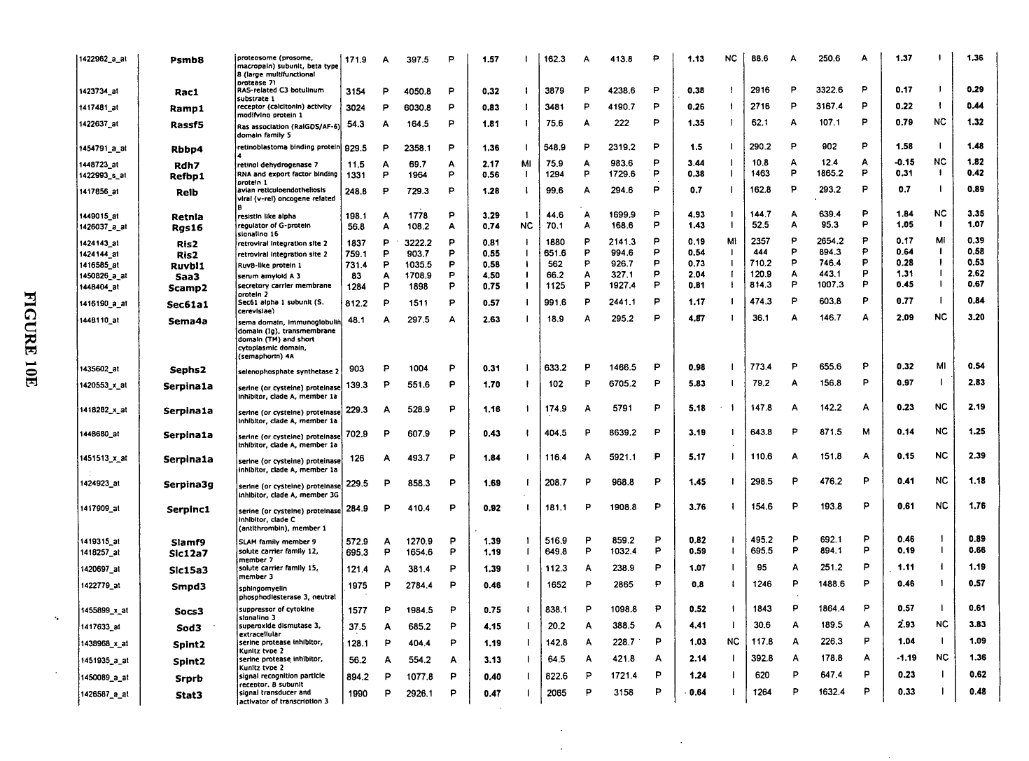

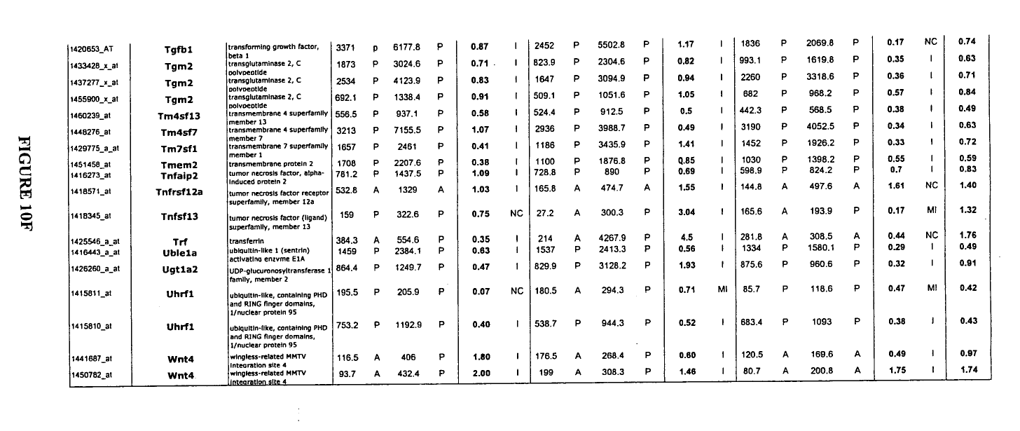

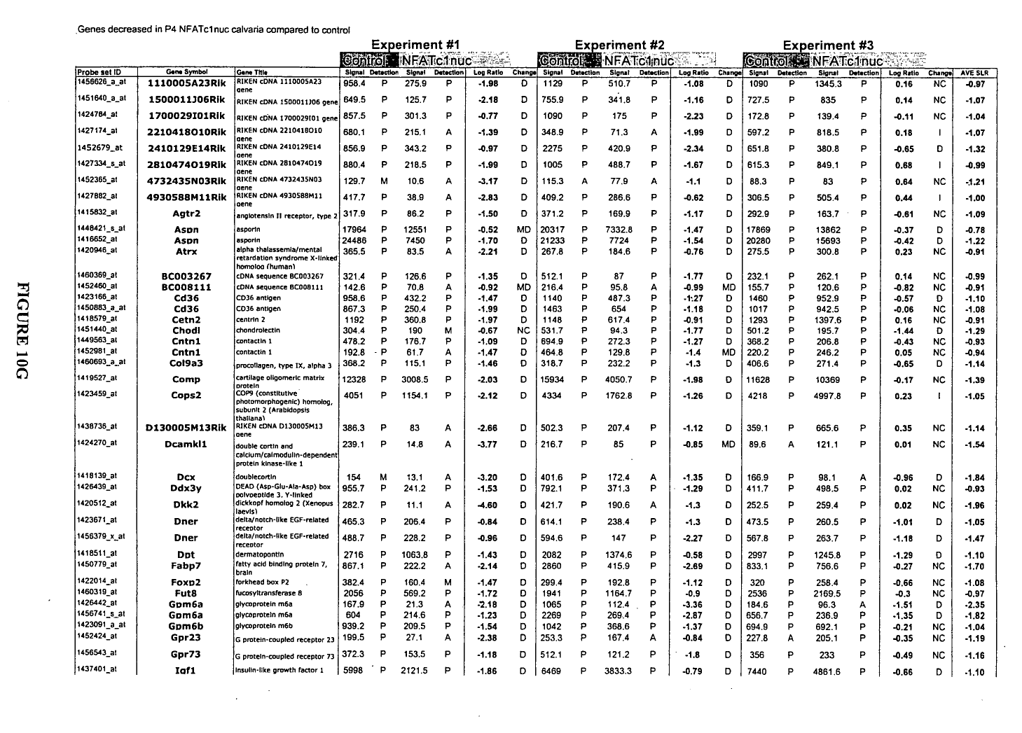

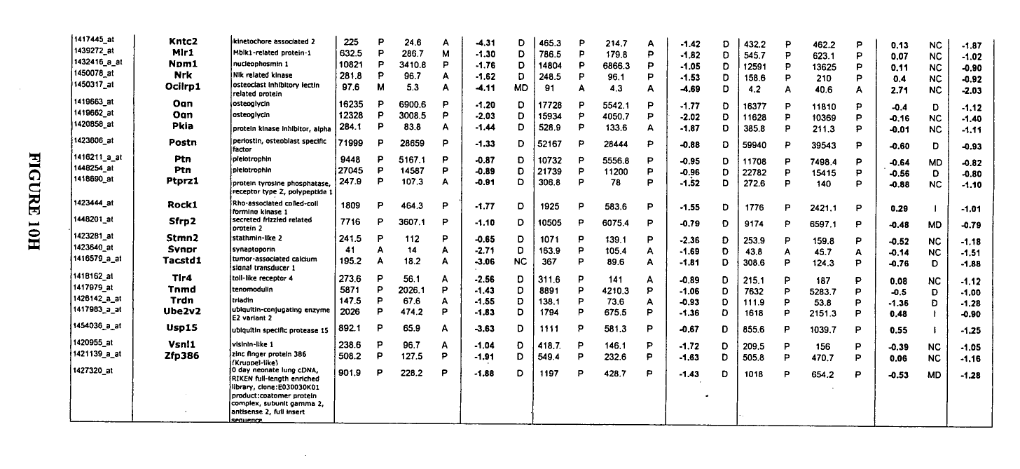

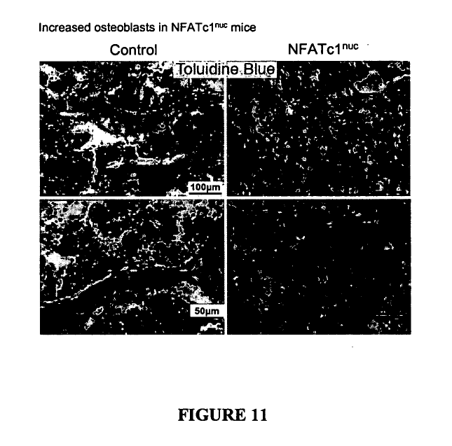

The invention also comprises a pharmaceutical composition comprising the use of an agent that regulates the activity of any one of the genes identified in FIGS. 9A, 9B and 10.

The pharmaceutical compositions of the invention may be formulated for administration in any convenient way for use in human or veterinary medicine. The pharmaceutical compositions of the invention include those suitable for oral, nasal, topical, and/or parenteral administration.

The phrase “pharmaceutically acceptable carrier” as used herein means a pharmaceutically acceptable material, composition or vehicle, such as a liquid or solid filter, diluent, adjuvant, excipient, solvent or encapsulating material, involved in carrying or transporting a subject drug from one organ, or portion of the body, to another organ, or portion of the body. Each carrier must be “acceptable” in the sense of being compatible with other ingredients of the formulation and not injurious to the patient. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

The pharmaceutical compositions of the invention may conveniently be presented in unit dosage form and may be prepared by any methods well known in the art of pharmacy. The amount of active ingredient(s) which can be combined with a carrier material to produce a single dosage form will vary depending upon the host being treated, the particular mode of administration. The amount of active ingredient(s) which can be combined with a carrier material to produce a single dosage form will generally be that amount of the agent(s) which produces a therapeutic effect.

### EXAMPLES

### Example 1

### Eμ-tTA/NFATc1nuc Mice have Increased Bone

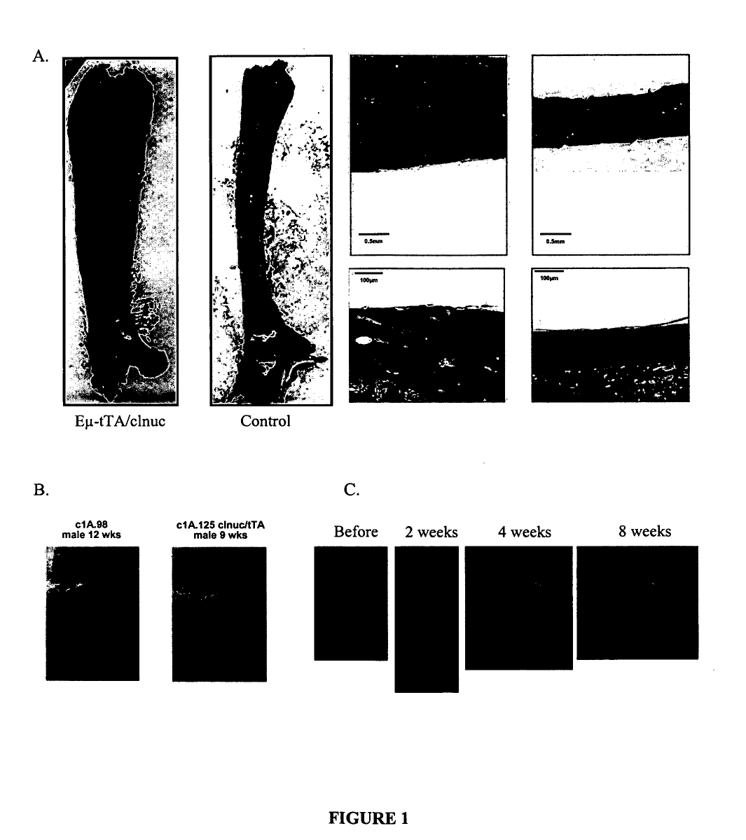

We created a line of transgenic mice that express a constitutively nuclear and constitutively active human NFATc1 (one of the four Nuclear Factor of Activated T-cell Transcription factors) (Beals et al., Gene and Development 1997 shows the in vitro activity of this mutant NFATc1). We put the transgene under the control of the tet-operator (tet-O) and crossed these mice into a line of mice that express the tet-transactivator (tTA) under the control of the IgM enhancer (Eμ) (Felsher et al., Mol. Cell, 1999). The Eμ-tTA/NFATc 1 nuc double transgenic mice have several interesting phenotypes. The mice have increased bone as assessed radiographically and histologically with an almost complete ablation of the bone marrow cavity in long bones as well as increased thickness of the ribs, spine, and skull (FIG. 1). This bone phenotype is independent of any contribution of T cells (where Eμ-tTA was reported to be expressed) because Eμ-tTA/C1nuc;TCRα−/− mice still have increased bone thickness (FIG. 1b).

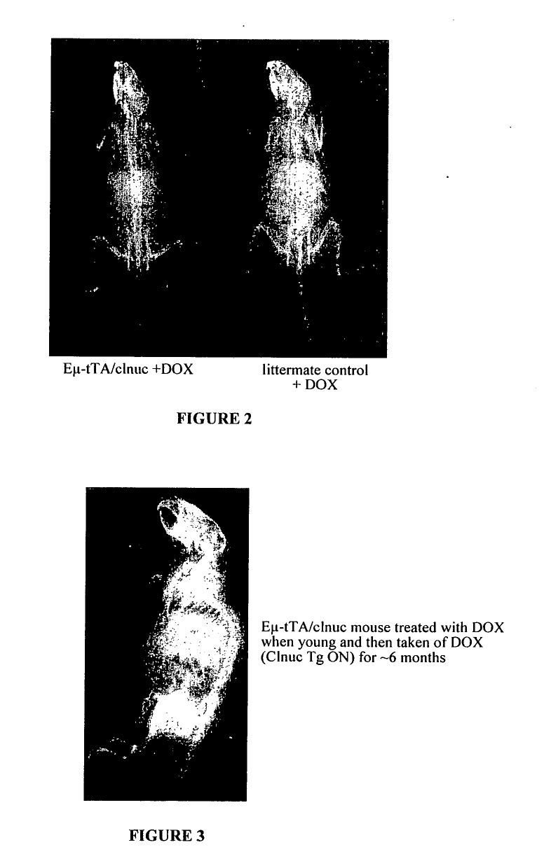

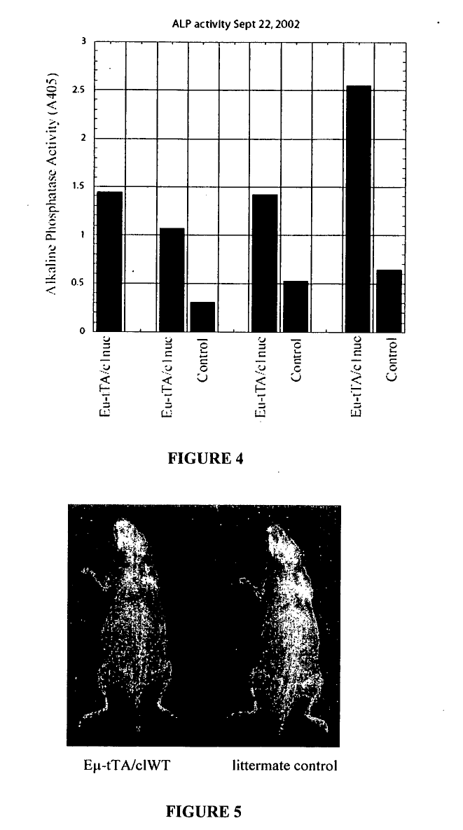

Our system allows NFAT c1nuc expression to be turned off by adding doxycycline to the drinking water. Treatment with doxycycline prevents the bone phenotype from developing (if given to the mice throughout life) (FIG. 2) or reverses the phenotype observed in the long bones (if started at 6 weeks of age) (FIG. 1c). Additionally, mice treated with doxycycline when young develop increased bone later in life after withdrawal from doxycycline and re-expression of the NFATc1nuc transgene (FIG. 3). This indicates that this pathway can function in adult osteoblasts and increase bone formation.

This phenotype required constitutively nuclear and constitutively active NFATc1 as transgenic expression of wild type NFATc1 does not result in this bone phenotype (FIG. 5). This is important because the expression of NFATc1 has been shown as a critical step in osteoclast development but here we find that the activation of NFATc1 is the critical step for increased osteoblast function in vivo.

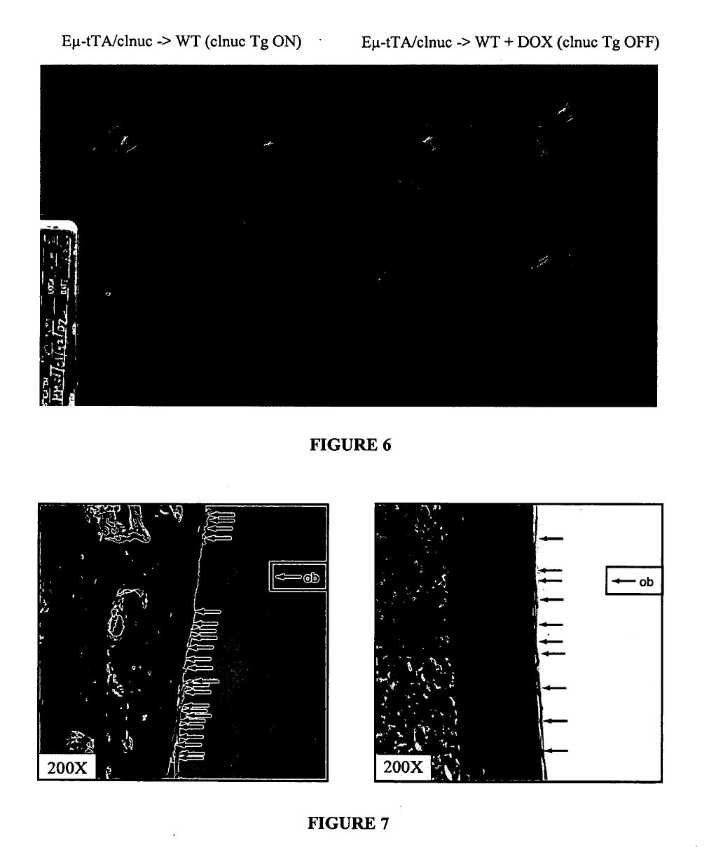

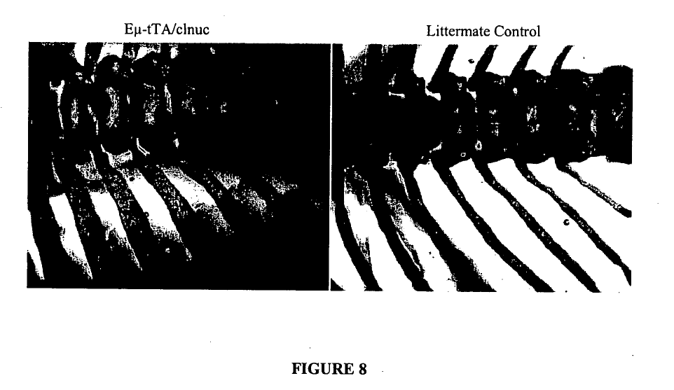

Several pieces of data demonstrate that this bone phenotype is due to the actions of NFATc1 in osteoblast: (1) An increase in serum alkaline phosphatase in Eμ-tTA/NFATc1nuc mice (FIG. 4); (2) Eμ-tTA/TetO-myc double transgenic mice using the same Eμ-tTA develop osteogenic sarcomas (Jain et al., Science, 2002) indicating that the Eμ-tTA may be expressed in osteoblasts; (3) Normal tooth eruption (arguing against an overall absence of osteoclast function) (data not shown); (4) Reconstitution of lethally irradiated wild-type mice with Eμ-tTA/NFATc1nuc bone marrow do not develop the bone phenotype despite the fact that the chimeric mice would be expected to have Eμ-tTA/NFATc1nuc osteoclasts (FIG. 6); (5) Increase in the number of osteoblasts by H+E staining on femora (FIG. 7); and (6) Increase in rib width in 2 day old Eμ-tTA/NFATc1nuc pups as assessed by alcian blue/alizarian red staining compared to non-transgenic littermate controls (FIG. 8).

### Example 2

### Genes Expressed Differentially in Eμ-tTA/NFATC1nuc Mice are Involved in Bone Formation

Isolation of RNA, hybridization and analysis of Microarrays: Four-day-old Eμ-tTA/NFATc1nuc and littermate control calvaria (4-6 calvaria per replicate, three replicates per group) were removed and lysed in Trizol reagent and RNA isolated according to the manufacturer protocol (Invitrogen). cDNA synthesis was performed with the SuperScript Choice synthesis kit (Gibco, 18090-019) using T7-(dT)24 primers (Operon). Biotin-labeled cRNA was synthesized using the Enzo BioArray kit (Affymetrix 900182) and fragmented according to manufacturer instructions. Hybridization to GeneChip® Mouse Genome 430 2.0 Array (Santa Clara, Calif.) and scanning of the chips were performed by the Stanford Affymetrix Core Facility. The scanned images were converted to numerical values with the GCOS software (Affymetrix) using the all-probe sets scaling strategy. Gene expression data analysis and comparisons between arrays was performed with the Data Mining Tool (DMT) software (Affymetrix) using the Wilcoxon Signed-Rank Test to compared paired cells for each probe set. A further validation of significant genes was achieved by running the Significance Analysis of Microarrays (SAM) program [PNAS2001 98: 5116-5121].

FIG. 10 shows gene expression changes in Eμ-Tta/NFATc1nuc calvaria: This table refers to the Affymetrix probe set ID, gene symbol, gene name and the signal for each feature as well as the Log Ratio for each of the three comparisons. The average SLR is shown in the right-most column and represents the log2 of the fold change. Genes for which at least three of the independent samples were called “present” or “marginal” and were changed (either increased or decreased) with a SLR>1 are shown. Detection is P (present), M (marginal), or A (absent). Changes is 1 (increased), MI (marginally increased), NC (not changed), MD (marginally decreased) or D (decreased).

Definition of Genes Misregulated in Eμ-tTA/NFATc1nuc calvaria: To learn more about the molecular changes induced by NFATc1nuc expression in osteoblasts global gene expression profiling was used to compare gene expression between control and NFATc1nuc P4 calvaria. We chose to analyze whole P4 calvaria in order to get a “snap-shot” of the total bone gene expression at a time point early in the development of this phenotype. We anticipated that two broad sets of genes would be differentially expressed. First, genes that are changed in osteoblasts as a direct consequence of NFATc Inuc expression and second, genes that are changed in response to the increased bone mass and likely represent genes that regulate the coupling between bone formation and bone resorption.

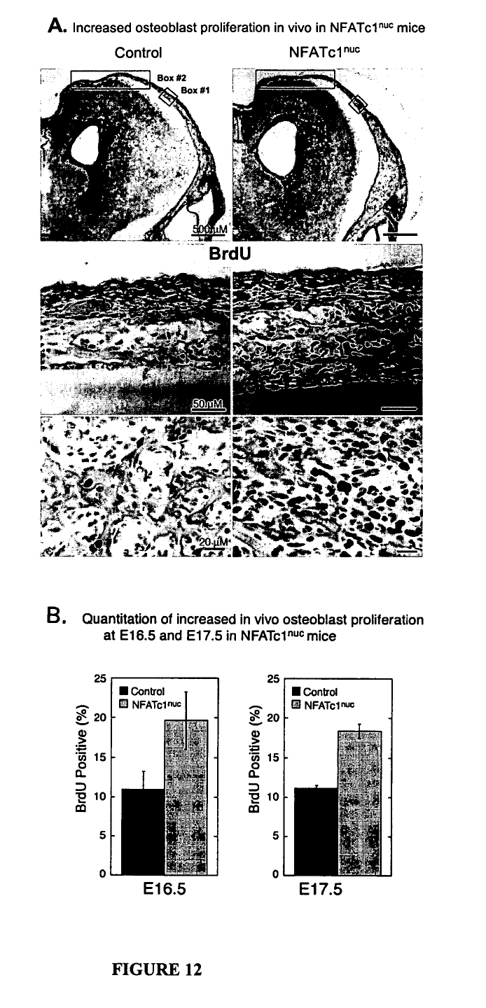

Four groups of genes were changed in the NFATc1nuc calvaria; osteoblast genes, cell cycle genes, chemoattractants, and monocytes genes (FIG. 9A and FIG. 10). First, many osteoblast functional genes, growth factors, and growth factors receptors were changed (FIG. 9A). These genes likely reflect a global change in the genetic program that drives the increased immature osteoblast proliferation observed in the Eμ-tTA/NFATc1nuc mice. Whether these genes are directly controlled by NFAT and their relative contribution to the phenotype, remain to be answered. Second, many genes that tightly regulate the G1 cell cycle checkpoint (Cks1, cyclin D1, Cdt1, and Cdk4) and S-phase progression (nuclear protein 95 and cyclin F) are increased in Eμ-tTA/NFATc1nuc calvaria (FIG. 9A). These genes represent a molecular confirmation of the in vivo BrdU labeling results (FIG. 12).

Interestingly, we found that several potential monocytes chemoattractants (CCL8/monocytes chemoattractant protein-2 (MCP-2), CCL6/C10, and CCL12/MCP-5) were increased in the Eμ-tTA/NFATC1nuc calvaria (FIG. 9B). These small chemotactic cytokines may function to recruit osteoclast progenitors to the bone and hence increase that number of potential bone resorbing osteoclasts. The most highly upregulated chemokine in the Eμ-tTA/NFATc1nuc calvaria, CCL8 (originally named monocytes chemoattractant protein-2), was cloned from an osteogenic sarcoma line and CCL8 and CCL12 have been shown to specifically attract monocytes in vitro and in vivo (van Damme et al, JEM, 1992; Sarafi et al, JEM, 1997; van Coillie et al, BBRC, 1997). The upregulation of this set of chemokines would oppose the dramatic increase in bone formation even at this early post-natal time point. This hypothesis is supported by the increase in many monocytes/osteoclast genes in the Eμ-tTA/NFATc1nuc calvaria. These included MHC class II molecules, complement components and FcγRIIb (FIG. 9B). Eμ-tTA/NFATc1nuc P4 calvaria also had increased TRAP staining (FIG. 9C).

The monocytes chemoattractant, CCL8 appears to be a direst NFAT target. The 5′ flanking region and introns of the CCL8 gene are highly conserved between multiple species and contain many possible NFAT binding sites. Therefore we inserted luciferase directly in-frame in the first exon to create a CCL8 reporter. This reporter was highly induced by PMA/Ionomycin stimulation and its activity was block with CsA (FIG. 9D). Collectively, our results indicate that NFATc1 in osteoblasts directly controls CCL8 expression and recruitment of monocytes osteoclast precursors to the bone.

NFATc1 Directs a Genetic Program of Osteoblast Proliferation: Analysis of the genes under control of NFATc1 in osteoblasts indicates that a discrete genetic program is set in motion when NFATc1 is activated and enters the osteoblast nucleus. NFATc1 directs, either directly or indirectly, the expression of Wnt4, Frizzled9, IGFBP1, PTH receptor, TPA and the repression of periostin and DKK2. Each of these genes, or homologues of them, is known to have critical roles in osteoblast proliferation or function. Stimulation of MC3T3-E1 cells was found to induce Wnt4 expression in a calcineurin dependent manner (FIG. 9E). Collectively, this in vivo and in vitro data indicates that Wnt4 can be regulated in osteoblasts by calcineurin/NFAT signaling. Eμ-tTA/NFATC1nuc mice also have increased expression of genes that directly control the G1-S transition and function during M phase. Most notably cyclin D1, which is critical for Rb phosphorylation and entry into S-phase is increased in Eμ-tTA/NFATC1nuc mice.

NFAT Directs Gene Expression that Induces Monocytes Recruitment: The NFATc family of transcription factors has been shown to control the communication between different cell types in the immune system as well as during cardiac development and angiogenesis (Crabtree and Olson, Cell, 2002). A possible role for the NFAT pathway in directing the tightly cross-regulated functions of bone forming and bone resorbing cells by controlling either soluble or cell surface proteins is intriguing.

Chemokine and integrin signals coordinate the spatially and temporally discrete patterns of cell migration in vivo (Butcher, Cell, 1991). A dramatic increase in the expression of several monocyte chemoattractants (CCL6, CCL8 and CCL12) in Eμ-tTAINFATc1nuc calvaria led us to test whether the calcineurin/NFAT pathway regulated these chemokines. We found that NFAT can control the expression of CCL8/MCP-2. The presence of a number of conserved NFAT binding sites in the promoter and intronic regions of CCL8 suggests that NFAT is a direct, rather than indirect, regulator of CCL8. CCL8, CCL12, and several other chemokines are located in a cluster on mouse chromosome 11 and therefore these monocyte chemoattractants may be coordinately expressed. Bone formation and bone resorption are integrated processes and the production of monocytes chemoattractant proteins by osteoblasts is an additional level of coordination (Parfill, Bone, 1998).

Thus the program of genes directed by a slight increase in NFATc1 nuclear occupancy nicely explains the phenotype of the Eμ-tTA/NFATc1nuc mice and provides evidence for an important coordinating mechanism entailing chemoattraction and control of differentiation.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method for (i) reducing loss of bone mass or bone density, (ii) increasing bone mass or bone density, (iii) maintaining bone mass or bone density and/or (iv) reducing loss of calcium from bone, comprising: administering to a subject a therapeutically effective amount of an NFAT agonist.

2. The method of claim 1 for the treatment of a bone condition or for promoting the healing of bone fractures.

3. The method of claim 2, wherein the bone condition being treated is osteoporosis.

4. The method of claim 1, wherein the NFAT agonist is administered conjointly with an agent that increases bone mass or bone density, or prevents the loss of bone mass or bone density.

5. A method for increasing osteoblast activity or decreasing osteoclast activity comprising the use of an NFAT agonist.

6. A pharmaceutical composition comprising an NFAT agonist and a pharmaceutically acceptable carrier.

7. A method for the bone tissue regeneration comprising the use of an NFAT agonist.

8. A method of determining whether an agent is an NFAT agonist which promotes, maintains, or reduces the loss of, bone mass or bone density comprising:
(a) determining whether the agent is an NFAT agonist; and further
(b) determining whether the agent promotes, maintains, or reduces the loss of, bone mass or bone density.

9. The method of claim 8, wherein determining whether the agent is an NFAT agonist comprises:
(a) contacting the agent with a cell comprising NFAT; and
(b) determining the location of NFAT within the cell in the presence and in the absence of the agent; wherein an increase of NFAT in the nucleus indicates that the agent is an NFAT agonist.

10. The method of claim 8, wherein determining whether the agent is an NFAT agonist comprises:
(a) contacting a cell expressing NFAT with an agent; and
(b) determining the phosphorylation state of NFAT in the presence and absence of the agent; wherein a decrease in the phosphorylation of NFAT indicates that the agent is an NFAT agonist.

11. The method of claim 8, wherein determining whether the agent is an NFAT agonist comprises:
(a) contacting NFAT with a phosphatase under conditions that allow the dephosphorylation of NFAT in the presence and in the absence of an agent, and
(b) determining the phosphorylation state of NFAT, wherein an decrease in the phosphorylation indicates that the agent is an NFAT agonist.

12. The method of claim 8, wherein determining whether the agent is an NFAT agonist comprises:
(a) contacting NFAT with a kinase under conditions that allow the phosphorylation of NFAT in the presence and in the absence of an agent; and
(b) determining the phosphorylation state of NFAT, wherein an decrease in the phosphorylation indicates that the agent is an NFAT agonist.

13. The method of claim 8, wherein determining whether the agent is an NFAT agonist comprises:
(a) transfecting a cell with an expression vector comprising a nucleic acid encoding a reporter gene operatively linked to an NFAT dependent transcriptional regulatory sequence;
(b) incubating the cell in the presence and absence of an agent; and
(c) measuring the expression of the reporter gene; wherein an increase in the expression of the reporter gene indicates that the agent is an NFAT agonist.

14. A method for identifying patients having or at risk of having a bone condition, which method comprises determining NFAT dependent transcriptional activity levels in osteoblasts isolated from the patients, and identifying those patients having an abnormally low level of NFAT dependent transcriptional activity as having or at risk of having a bone condition.

15. A method to screen for target genes that increase osteoblast activity or decrease osteoclast activity:
(a) identifying a target gene whose expression is regulated by NFAT, and
(b) determining whether the regulation of the expression of the target gene identified in step (a) increases osteoblast activity or decreases osteoclast activity.

16. A method to screen for target genes that reduce the loss of bone mass, reduce the loss of bone density, and/or reduce loss of calcium from bone comprising:
(a) identifying a target gene whose expression is regulated by NFAT, and
(b) determining whether the regulation of the expression of the target gene identified in step (a) reduces loss of bone density, reduces the loss of bone mass, and/or reduces loss of calcium from bone in vivo.

17. A method to screen for agents that increase osteoblast activity or decreases osteoclast activity comprising:
(a) identifying a target gene whose expression is regulated by NFAT,
(b) determining whether the regulation of the expression of the target gene identified in step (a) increases osteoblast activity or decreases osteoclast activity, and
(c) further identifying an agent that mimics or agonizes the activity of the target gene.

18. A method to screen for agents that reduce the loss of bone mass, reduce the loss of bone density or reduce loss of calcium from bone comprising:
(a) identifying a target gene whose expression is regulated by NFAT,
(b) determining whether the regulation of the expression of the target gene identified in step (a) reduces loss of bone density, reduces bone mass, and/or reduces loss of calcium from bone in vivo, and
(c) further identifying an agent that mimics or agonizes the activity of the target gene.

19. A method for (i) reducing loss of bone mass or bone density, (ii) increasing bone mass or bone density, (iii) maintaining bone mass or bone density and/or (iv) reducing loss of calcium from bone, comprising the use of an agent that regulates at least one of the genes identified in FIGS. 9A, 9B or 10.

20. A method for increasing osteoblast activity or decreasing osteoclast activity comprising the use of an agent that regulates at least one of the genes identified in FIGS. 9A, 9B or 10.
